(19) United States
(12) Patent Application Publication
(10) Pub. No.: US 2024/0108727 A1
(43) Pub. Date: Apr. 4, 2024

(54) EUTECTIC MATRIX FOR NUTRACEUTICAL COMPOSITIONS

(71) Applicant: InovoBiologic, Inc., Calgary (CA)

(72) Inventors: Roland Jacques Gahler, Burnaby (CA); Simon Wood, Victoria (CA); Chuck Chang, Coquitlam (CA); Yun Chai Kuo, Vancouver (CA)

(73) Assignee: InovoBiologic, Inc., Calgary, AB (CA)

(21) Appl. No.: 18/255,289

(22) PCT Filed: Dec. 2, 2021
(86) PCT No.: PCT/CA2021/051722

§ 371 Date: May 31, 2023

Related U.S. Application Data
(60) Provisional application No. 63/121,714, filed on Dec. 4, 2020.

(51) Int. Cl. A61K 47/20; A61K 31/352; A61K 47/10; A61K 47/14

(52) U.S. Cl. CPC A61K 47/20; A61K 31/352; A61K 47/10; A61K 47/14

(57) ABSTRACT

The present disclosure describes compositions including a eutectic matrix that includes methylsulfonylmethane and a sugar alcohol and particles of a nutraceutical in the eutectic matrix. In some embodiments, the sugar alcohol:methylsulfonylmethane ratio may be from 95:5 to 20:80. Also provided herein are methods for making the compositions and methods for increasing solubility and bioavailability of a nutraceutical.

### BACKGROUND

Flavonoids or bioflavonoids are polyphenolic compounds found in plants and fungi. Many flavonoids are hydrophobic and have poor solubility in water. As a result, they have poor bioavailability.

For example, flavonols are a subclass of flavonoids with a molecular structure based on a 3-hydroxyflavone backbone. They are widely distributed in nature and are most commonly found in fruits and vegetables. Some examples of common flavonols include fisetin, galangin, gossypetin, kaempferide, kaempferol, isorhamnetin, morin, myricetin, pachypodol, quercetin, rhamnazin, rhamnetin and others. While flavonols have limited solubility in water, glycosides of flavonols have somewhat better solubilities.

As another example, quercetin is one of the most commonly consumed flavonoid compounds from daily diets. It's been reported to be found in food items such as capers, dill, cilantro, onions, kale, chokeberry cranberry, and plums. Quercetin is generally present as quercetin glycoside in nature and involves quercetin aglycone conjugated to sugar moieties such as glucose or rutinose. Quercetin has been reported to exhibit antioxidative, anti-carcinogenic, anti-inflammatory, anti-aggregatory and vasodilating effects. Unfortunately, quercetin bioavailability is generally poor, and several factors affect its bioavailability.

Examples of other poorly water-soluble compounds having poor bioavailability include quercetin glycosides such as rutin (a rutinose glycoside of quercetin, found, for example, in capers, raspberries, buckwheat, and asparagus); and curcuminoids, which are generally hydrophobic compounds thought to have a variety of therapeutic benefits such as anti-inflammatory, anti-oxidant, and/or anti-cancerous activities. Additional examples include oil soluble vitamins, such as vitamin A, D2, D3, E, or K, isolated dietary compounds containing non-polar moieties such as sterols, stanols, omega-3 fatty acids or esters of omega-3 fatty acids sourced from fish, krill, plant or algae, or carotenoids such as astaxanthin, lutein and zeaxanthin.

However, their therapeutic use has been limited by their hydrophobicity which results in poor solubility and rapid elimination from the body (i.e., low bioavailability).

There is a need for solubilizing matrices that enhance solubilization and bioavailability of flavonoids or bioflavonoids. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a composition including a eutectic matrix comprising methylsulfonylmethane and a sugar alcohol; and particles of a nutraceutical in the eutectic matrix.

In one embodiment, the sugar alcohol:methylsulfonylmethane ratio is from 95:5 to 20:80. In one embodiment, the sugar alcohol is a 4-carbon sugar alcohol, a 5-carbon sugar alcohol, a 6-carbon sugar alcohol, a 12-carbon sugar alcohol, or any combination thereof. In one embodiment, the sugar alcohol is selected from erythritol, xylitol, arabitol, ribitol, mannitol, sorbitol, galactitol, fucitol, iditol, inositol, maltitol, and any combination thereof. In one embodiment, the sugar alcohol is xylitol.

In one embodiment, the eutectic matrix has a melting point lower than each of the melting point of the methylsulfonylmethane and the melting point of the sugar alcohol. In one embodiment, the eutectic matrix is methylsulfonylmethane and the sugar alcohol. In one embodiment, the methylsulfonylmethane and the sugar alcohol form an organized structure or a repeating pattern. In one embodiment, the nutraceutical forms hydrogen bonds and/or ionic bonds with the eutectic matrix.

In one embodiment, the nutraceutical is quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof. In one embodiment, the nutraceutical is quercetin.

In one embodiment, the composition further includes a lipid. In one embodiment, the lipid is an edible oil or a mixture of oils. In one embodiment, the lipid is medium chain triglycerides. In one embodiment, the lipid forms a lipid soluble matrix with the methylsulfonylmethane, the sugar alcohol, the nutraceutical. In one embodiment, the composition further includes saponins, cocoa, or any combination thereof.

In one embodiment, the composition has an AUC_{0-8 hours} of greater than 250 ng/ml of the nutraceutical when administered to a subject. In one embodiment, the composition is in the form of a tablet, a capsule, a softgel, a gummy, or a liquid.

In one embodiment, the nutraceutical has a water solubility at least 4% greater compared to the water solubility of the nutraceutical in each individual matrix component. In one embodiment, the nutraceutical has a water solubility not linearly correlated to a diameter of the particles or not linearly correlated with a percentage of methylsulfonylmethane in the composition. In one embodiment, the nutraceutical has a peak water solubility at a methylsulfonylmethane concentration of from 20% to 50% by weight. In one embodiment, the nutraceutical has a water solubility greater than the sum of the water solubilities of the nutraceutical in each of methylsulfonylmethane and sugar alcohol. In one embodiment, the composition has an enteric coating.

In one aspect, the present disclosure provides a method for making a composition by mixing methylsulfonylmethane and a sugar alcohol to form a mixture; heating the mixture to melt the methylsulfonylmethane and a sugar alcohol; adding a nutraceutical composition; and cooling the mixture to form a eutectic matrix with particles of the nutraceutical in the eutectic matrix.

In one aspect, the present disclosure provides a method for making a composition by dissolving methylsulfonylmethane and a sugar alcohol in water, ethanol, acetone, or a polar solvent to form a solution; adding a nutraceutical to the solution; and re-crystallizing the solution to obtain a eutectic matrix with particles of the nutraceutical in the eutectic matrix.

In one aspect, the present disclosure provides a method for enhancing the solubility of a nutraceutical by adding a nutraceutical to a lipid-soluble matrix. In one embodiment, the lipid-soluble matrix includes methylsulfonylmethane, a sugar alcohol, and a lipid. In one embodiment, the methylsulfonylmethane and the sugar alcohol form a eutectic matrix. In one embodiment, the sugar alcohol:methylsulfonylmethane ratio is from 95:5 to 20:80

In one embodiment, the sugar alcohol is a 4-carbon sugar alcohol, a 5-carbon sugar alcohol, a 6-carbon sugar alcohol, a 12-carbon sugar alcohol, or any combination thereof. In one embodiment, the sugar alcohol is selected from erythritol, xylitol, arabitol, ribitol, mannitol, sorbitol, galactitol, fucitol, iditol, inositol, maltitol, and any combination thereof. In one embodiment, the sugar alcohol is xylitol.

In one embodiment, the lipid is an edible oil or a mixture of oils. In one embodiment, the lipid is medium chain triglycerides.

In one embodiment, the nutraceutical is quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof. In one embodiment, the nutraceutical is quercetin. In one embodiment, the lipid-soluble matrix further includes saponins, cocoa, or any combination thereof.

In one aspect, the present disclosure provides a method for enhancing the bioavailability of a nutraceutical by adding a nutraceutical to a lipid-soluble matrix.

In one embodiment, the lipid-soluble matrix includes methylsulfonylmethane, a sugar alcohol, and a lipid. In one embodiment, the methylsulfonylmethane and the sugar alcohol form a eutectic matrix. In one embodiment, the sugar alcohol:methylsulfonylmethane ratio is from 95:5 to 20:80. In one embodiment, the sugar alcohol is a 4-carbon sugar alcohol, a 5-carbon sugar alcohol, a 6-carbon sugar alcohol, a 12-carbon sugar alcohol, or any combination thereof. In one embodiment, the sugar alcohol is selected from erythritol, xylitol, arabitol, ribitol, mannitol, sorbitol, galactitol, fucitol, iditol, inositol, maltitol, and any combination thereof. In one embodiment, the sugar alcohol is xylitol.

In one embodiment, the lipid is an edible oil or a mixture of oils. In one embodiment, the lipid is medium chain triglycerides.

In one embodiment, the nutraceutical is quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof. In one embodiment, the nutraceutical is quercetin. In one embodiment, the lipid-soluble matrix further includes saponins, cocoa, or any combination thereof.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a graph of average quercetin concentrations over time in whole blood for an embodiment of a composition of the present disclosure and for comparative compositions.

FIG. 2A is a table of average quercetin concentrations over time in whole blood for a comparative composition containing quercetin as a micronized powder.

FIG. 2B is a table of average quercetin concentrations over time in whole blood for a comparative composition containing quercetin solubilized in a micellar matrix.

FIG. 2C is a table of average quercetin concentrations over time in whole blood for a comparative composition containing quercetin as a granulated powder.

FIG. 2D is a table of average quercetin concentrations over time in whole blood for a comparative composition containing quercetin solubilized in a micellar matrix containing peppermint oil.

FIG. 2E is a table of average quercetin concentrations over time in whole blood for a comparative composition in a hard gelatin capsule.

FIG. 2F is a table of average quercetin concentrations over time in whole blood for a comparative composition in a hard gelatin capsule.

FIG. 2G is a table of average quercetin concentrations over time in whole blood for a comparative composition in a hard gelatin capsule.

FIG. 2H is a table of average quercetin concentrations over time in whole blood for an embodiment of a composition of the present disclosure.

FIG. 2I is a table of average quercetin concentrations over time in whole blood for a comparative composition containing quercetin enzymatically modified with sugars.

FIG. 2J is a table of average quercetin concentrations over time in whole blood for a comparative composition containing quercetin formulated with cyclodextrin.

FIG. 3 is a high-pressure liquid chromatogram of the water solubility of epimedium extract. Black trace: without MSM:xylitol matrix. Blue trace: with MSM:xylitol matrix.

FIG. 4 is a high-pressure liquid chromatogram of the water solubility of curcuminoids. Black trace: without MSM:xylitol matrix. Blue trace: with MSM:xylitol matrix.

FIG. 5 is a high-pressure liquid chromatogram of the water solubility of echinacea alkylamides. Black trace: without MSM:xylitol matrix. Blue trace: with MSM:xylitol matrix.

FIG. 6 is a high-pressure liquid chromatogram of the water solubility of soy isoflavones. Black trace: without MSM:xylitol matrix. Blue trace: with MSM:xylitol matrix.

FIG. 7 is a high-pressure liquid chromatogram of the water solubility of hesperidin. Black trace: without MSM:xylitol matrix. Blue trace: with MSM:xylitol matrix.

FIG. 8 is a high-pressure liquid chromatogram of the water solubility of grapeseed extract. Black trace: without MSM:xylitol matrix. Blue trace: with MSM:xylitol matrix.

FIG. 9 is a high-pressure liquid chromatogram of the water solubility of milk thistle extract. Black trace: without MSM:xylitol matrix. Blue trace: with MSM:xylitol matrix.

FIG. 10 is a graph of non-linear behavior of quercetin solubility and hydrodynamic particle volume (particle size) as a function of MSM Ratio. Nutraceutical solubility not correlated to smaller particle size.

FIG. 11A is a table of melting points of embodiments of matrices of the present disclosure with measured melting points of varying ratios of MSM:xylitol.

FIG. 11B is a graph of melting points of embodiments of matrices of the present disclosure.

FIG. 12 shows tables of water solubilities of embodiments of compositions of the present disclosure.

FIG. 13 is a bar graph of solubilities of nutraceuticals in compositions of the present disclosure.

FIG. 14 is a graph of solubilities of embodiments of compositions of the present disclosure as a function of MSM ratio.

FIG. 15A is a table of solubilities of an embodiment of compositions of the present disclosure as a function of MSM ratio where the active constituent is grapeseed extract.

FIG. 15B is a graph of grapeseed extract catechin solubility as a function of MSM ratio.

FIG. 16 is a table of embodiments of quercetin compositions and their corrected solubilities in water.

FIG. 17A is a table of embodiments of epimedium compositions and their solubilities in water.

FIG. 17B is a graph of embodiments of epimedium compositions and their solubilities in water.

FIG. 18A is a table of embodiments of hesperidin compositions and their solubilities in water.

FIG. 18B is a graph of embodiments of hesperidin compositions and their solubilities in water.

FIG. 19A is a table of embodiments of curcumin compositions and their solubilities in water.

FIG. 19B is a graph of embodiments of curcumin compositions and their solubilities in water.

FIG. 20A is a table of pharmacokinetic properties of a comparative quercetin composition.

FIG. 20B is a table of pharmacokinetic properties of a composition of the present disclosure.

FIG. 20C is a graph of a concentration of quercetin in blood for a composition of the present disclosure and a comparative composition.

FIG. 21A is a table comparing the solubility of milk thistle extract in an unmodified form, in a eutectic matrix, and in a lipid-soluble matrix form.

FIG. 21B is a table comparing the solubility and bioavailability of berberine extract in an unmodified form, with ascorbic acid, and in a eutectic matrix with and without ascorbic acid.

FIG. 21C is a table comparing the solubility and permeability of vitamin D3 in an unmodified form and in a lipid-soluble matrix form.

FIG. 21D is a table comparing the bioavailability of boswellia extract in an unmodified form and in a lipid-soluble matrix with saponin form.

FIG. 21E is a table comparing the solubility of coenzyme Q10 (CoQ10) in an unmodified form and in a lipid-soluble matrix with cocoa form.

FIG. 21F is a table comparing the solubility of ubiquinol in an unmodified form and in a lipid-soluble matrix form with and without cocoa.

### DETAILED DESCRIPTION

The present disclosure features a composition, including a eutectic matrix that includes methylsulfonylmethane and a sugar alcohol; and a nutraceutical in the eutectic matrix. The nutraceutical can be in the form of particles. In some embodiments, the composition does not include phospholipids, sugars, oils, enzymes, caffeine, green tea, green tea extract, and/or vitamins.

### Definitions

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

It will be readily understood that the aspects of the present disclosure, as generally described herein, and illustrated in the figures, can be arranged, substituted, combined, separated, and designed in a wide variety of different configurations, all of which are explicitly contemplated herein.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means+/−5%. As used herein, recited ranges include the end points, such that from 0.5 mole percent to 99.5 mole percent includes both 0.5 mole percent and 99.5 mole percent.

The term “curcuminoid” as used herein, is meant to encompass a variety of linear diarylheptanoid compounds of natural and synthetic origins or even compounds of natural origin that are subsequently synthetically modified. A diarylheptanoid consists of two aromatic rings (aryl groups) joined by a seven carbons chain (heptane) or seven carbons forming a ring and linear linker (for example, see cyclocurcumin) and allowing for various substituents. Common curcuminoids may include one or more of the following: curcumin; demethoxycurcumin; bisdemethoxy curcumin; cyclocurcumin; tetrahy drocurcumin; dihy drocurcumin; curcumin-glucuronoside; dihydro curcumin-glucuronoside; tetrahy drocurcumin-glucuronoside; curcuminsulphate; and hexahydrocurcumin.

The term “green tea extract” as used herein is meant to encompass a tea extract from Camellia sinensis. For example, powdered decaffeinated green tea extract is available in numerous forms and purities from Millipore Sigma™ (i.e., CAS Number: 84650-60-2). The primary ingredient of which is epigallocatechin gallate (EGCG), is most abundant catechin in tea (i.e., for example, CAS Number 989-51-5).

The term “rutin” or “rutoside” as used herein is refers to the glycoside combining the flavonol quercetin and the disaccharide rutinose (α-L-rhamnopyranosyl-(1→6)-β-D-glucopyranose). It is a citrus flavonoid found in wide variety of plants including citrus fruit.

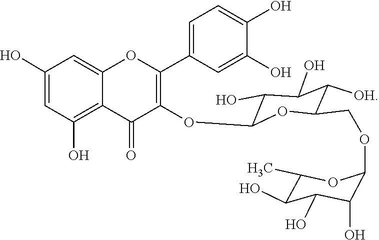 [SMILES: CC1OC(OCC2OC(Oc3c(-c4ccc(O)c(O)c4)oc4cc(O)cc(O)c4c3=O)C(O)C(O)C2O)C(O)C(O)C1O]

The term “methylsulfonylmethane” (MSM) as used herein refers to an organosulfur compound with the formula (CH₃)₂SO₂. MSM is also known by several other names including DMSO₂, methyl sulfone, and dimethyl sulfone. The structure of MSM is

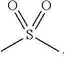

The term “sugar alcohol,” also referred to as a polyhydric alcohol, polyalcohol, alditol, or glycitol, refers to are organic compounds, typically derived from sugars, containing one hydroxyl group (—OH) attached to each carbon atom. Sugar alcohols have the general formula HOCH₂(CHOH)ₙCH₂OH.

The term “solubilization matrix” or “matrix,” as used herein is meant to encompass a particular subset of solubilizers or a single solubilizer.

The term “eutectic” refers to a homogeneous mixture of substances that melts or solidifies at a single temperature that is lower than the melting point of any of the constituents. The “eutectic temperature” is the lowest possible melting temperature over all of the mixing ratios for the involved component species.

The terms “solubilizer” as used herein refers to a substance that improves the solubility of a bioflavonoid in water.

The term “solubility” as used herein refers to the property of a solid, liquid, or gaseous chemical substance (“solute”) to dissolve in a solid, liquid, or gaseous solvent. The solubility of a substance fundamentally depends on the physical and chemical properties of the solute and solvent as well as on temperature, pressure and the pH of the solution. Solubility testing of the compositions of the present disclosure may proceeded as follows: 25-500 mg of a composition containing the equivalent of about 25 mg of the nutraceutical is added to 10 mL of distilled water in a screw cap test tube. The test tube is vortexed for 2 minute and sonicated in a water bath set to the specified temperature for 1 hour. Then samples were filtered 0.45 μm syringe filter and then analyzed by high-pressure liquid chromatography (HPLC). The filtrate after the filtration step contains water-soluble particles smaller than 450 nm. If the concentration determined by HPLC is not within the calibration curve, the sample is further diluted to a suitable concentration and re-analyzed.

The term “organic solvent” as used herein is refers to any solvent having at least 1 carbon atom and 1 hydrogen atom, a low molecular weight, lipophilicity, and volatility, and exist in liquid form at room temperature. Organic solvents may further be grouped as aliphatic or aromatic. Organic solvents are useful because they can dissolve oils, fats, resins, rubber, and plastics. An organic solvent may be selected from: methanol, ethanol, propanol, butanol, acetonitrile, and acetone.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the term “bioavailability” means the proportion of a drug or other substance, for example a nutraceutical, that enters the circulation when introduced into a subject and is able to have an active effect. Bioavailability testing of the compositions of the present disclosure may proceeded as follows. After an overnight fast, blood samples can be collected from each volunteer before ingesting the composition being tested. Blood can be again collected at 0.5 hours, 1 hour, 2 hours, 3 hours, 4 hours, 6 hours, and 8 hours. After all blood samples are collected, the blood samples can be processed as follows.
(1) Pipette a fix amount of blood (e.g., 50-100 μL) into a microcentrifuge vial;
(2) Add sufficient amount of enzyme (e.g., 100 μL) into the vial;
(3) Incubate the vial in the 37° C. for 1 hour to hydrolyze the nutraceutical conjugates;
(4) Add 10 μl of an Internal Standard (IS) working solution and 400 μl of a suitable solvent (typically ethanol, methanol, acetone, or isopropanol) to the vial;
(5) Sonicate the mixture for 10 minutes to 1.5 hours at 25°−50° C. to extract the analytes;
(6) Centrifuge at 3000 g or higher for 5 minutes and then place the supernatant in a suitable container for subsequent analysis,
(7) Run liquid chromatography mass spectrometry (LC-MS) analysis and analyze for blood concentrations of the nutraceutical. AUC values are calculated by integrating the area under the curve for the average blood concentrations over time for the product being tested.

As used herein, the phrase “therapeutically effective amount” refers to the amount of a therapeutic agent (i.e., drug, nutraceutical, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

“About” includes all values having substantially the same effect, or providing substantially the same result, as the reference value. Thus, the range encompassed by the term “about” will vary depending on context in which the term is used, for instance the parameter that the reference value is associated with. Thus, depending on context, “about” can mean, for example, ±15%, ±10%, ±5%, ±4%, ±3%, ±2%, ±1%, or ±less than 1%. Importantly, all recitations of a reference value preceded by the term “about” are intended to also be a recitation of the reference value alone. Notwithstanding the preceding, in this application the term “about” has a special meaning with regard to pharmacokinetic parameters, such as area under the curve (including AUC, AUCₜ, and AUC_{∞}) Cₘₐₓ, Tₘₐₓ, and the like. When used in relationship to a value for a pharmacokinetic parameter, the term “about” means from 80% to 125% of the reference parameter.

“Nutraceutical” or “nutraceutical composition” refers to a formulation of a compound of the disclosure, such as a botanical extract containing one or more phytochemicals such flavonoids, an isolated dietary compound containing a non-polar moiety such as sterols, stanols, quinones, carotenoids, terpenes, or fatty acids, or a vitamin, and a matrix generally accepted in the art for the delivery of the nutraceutical to mammals, e.g., humans. Such a medium includes all pharmaceutically acceptable carriers, diluents or excipients therefor.

“Pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Enteric polymer” refers to a polymer that is poorly soluble in aqueous medium at a pH of about 4.5 or less but becomes soluble in aqueous medium at a pH of greater than about 5. For example, an enteric polymer is poorly soluble in gastric juice, but is soluble in the lower GI tract environment.

### Compositions

As described above, the compositions of the present disclosure include a eutectic solubilizing matrix including methylsulfonylmethane (MSM) and a sugar alcohol; and a nutraceutical in the eutectic matrix.

In some embodiments, the sugar alcohol:methylsulfonylmethane ratio is from 95:5 (e.g., from 90:10, from 80:20, from 70:30, from 60:40, from 50:50, from 40:60, from 30:70) to 20:80 (e.g., to 30:70, to 40:60, to 50:50, to 60:40, to 70:30, to 80:20, to 90:10) by weight.

In certain embodiments, the sugar alcohol:methylsulfonylmethane ratio is from 25:75 to 70:30 by weight. In preferred embodiments, the sugar alcohol:methylsulfonylmethane ratio is from 40:60 to 65:35 by weight.

The sugar alcohol can include a 4-carbon sugar alcohol, a 5-carbon sugar alcohol, a 6-carbon sugar alcohol, a 12-carbon sugar alcohol, or any combination thereof. In some embodiments, the sugar alcohol is erythritol, xylitol, arabitol, ribitol, mannitol, sorbitol, galactitol, fucitol, iditol, inositol, and/or maltitol. In certain embodiments, the sugar alcohol is xylitol.

The eutectic matrix (i.e., eutectic solubilizing matrix) has a melting point lower than the melting point of the methylsulfonylmethane and the melting point of the sugar alcohol(s) in the eutectic matrix. In some embodiments, the eutectic matrix consists of methylsulfonylmethane and the sugar alcohol. In some embodiments, the eutectic matrix consists of methylsulfonylmethane and xylitol. The range of the ratios of the methylsulfonylmethane to sugar alcohol in a eutectic matrix can be determined by determining the melting point of the combined mixture. Briefly, MSM and a sugar alcohol can be mechanically mixed together. Then the mixture can be heated at gradually increasing temperatures until melting is observed and heating is stopped when the mixture is completely melted. Alternatively, the mixture of MSM and a sugar alcohol can be dissolved in water, ethanol, acetone, or a suitable polar solvent, or in a combination of polar solvents. The solution is then re-crystalized or dried by spray-drying, vacuum-drying, freeze-drying, or by other drying techniques to obtain a homogeneous mixture of MSM and the sugar alcohol in solid form. It is also possible to melt MSM or the sugar alcohol so that one component becomes a liquid. The not-yet-melted solid component is then dissolved in the liquid component and mixed as a homogeneous mixture. The mixture solidifies upon cooling to provide a solid mixture. The solid mixture can then be subjected to melting-point determination. If the determined melting point of the solid mixture is the below the melting point of the lower melting component, or lower than the theoretical melting point calculated from a proportional arithmetic combination of the melting points, then the mixture is a eutectic matrix. In some embodiments, the range of the ratios of the methylsulfonylmethane to xylitol in a eutectic matrix is from 5:95 to 80:20 by weight.

The methylsulfonylmethane and the sugar alcohol can form an organized structure, such as a repeating pattern. In some embodiments, the nutraceutical forms hydrogen bonds and/or ionic bonds with the eutectic matrix. In some embodiments, the nutraceutical, the methylsulfonylmethane, and the sugar alcohol together form an organized structure, such as a repeating pattern. For example, the repeating pattern can occur in a crystalline solid. In some embodiments, an organized structure of the nutraceutical, the methylsulfonylmethane, and the sugar alcohol can be suspended in a liquid.

In some embodiments, the nutraceutical includes a flavonoid and/or a bioflavonoid, such as quercetin, epimedium extract (e.g., an epimedium extract obtained by extracting epimedium with an organic solvent such as an alcohol or ketone or a mixture of an organic solvent with water), curcuminoid, echinacea alkylamides (e.g., isolated as described in U.S. Pat. No. 6,511,683, incorporated herein by reference in its entirety), soy isoflavones (e.g., genistein, genistin, malonyl genistin, acetyl genistin, daidzein, daidzin, malonyl daidzin, acetyl daidzin, glycitein, glycitin, malonyl glycitin, and/or acetyl glycitin), hesperidin, grapeseed extract (e.g., a grapeseed extract obtained by extracting grapeseed with an organic solvent such as an alcohol or ketone or a mixture of an organic solvent with water, or extracted with hot water or steam), milk thistle extract (e.g., obtained by extracting milk thistle with an organic solvent such as an alcohol or ketone or a mixture of an organic solvent with water), procyanidins (e.g., obtained from cocoa liquor), schisandra berry extract (e.g., obtained by extracting schisandra berry with an organic solvent such as an alcohol or ketone or a mixture of an organic solvent with water), lemon balm extract (e.g., obtained by extracting lemon balm with an organic solvent such as an alcohol or ketone or a mixture of an organic solvent with water), ginger root extract (e.g., obtained by extracting ginger root with supercritical carbon dioxide or an organic solvent), rhodiola extract (e.g., obtained by extracting rhodiola with an organic solvent such as an alcohol or ketone or a mixture of an organic solvent with water), berberine extract (e.g., obtained by extracting Berberis vulgaris with an organic solvent such as an alcohol or ketone or a mixture of an organic solvent with water), and/or boswellia extract (e.g., obtained by extracting boswellia with an organic solvent such as an alcohol or ketone or a mixture of an organic solvent with water). In some embodiments, the nutraceutical includes quercetin.

In some embodiments, the nutraceutical includes a quinone, such as pyrroloquinoline quinone, CoQ10 and/or ubiquinol. In some embodiments, the nutraceutical includes oil soluble vitamins, such as vitamin A, D2, D3, E, or K. In some embodiments, the nutraceutical includes isolated dietary compounds containing non-polar moieties such as sterols, stanols, omega-3 fatty acids or esters of omega-3 fatty acids sourced from fish, krill, plant or algae, or carotenoids such as astaxanthin, lutein and zeaxanthin.

In some embodiments, the particles of the nutraceutical are mixed with a lipid to form a lipid-soluble matrix. In some embodiments, the lipid comprises a fat selected from the group consisting of a dairy fat (e.g., milk fat, butter fat), an animal fat (e.g., lard) or a vegetable fat (e.g., coconut oil, cocoa butter, or palm oil).

In some embodiments, the lipid comprises an edible oil or a mixture of oils. Such oils include vegetable oils (e.g., canola oil, soybean oil, palm kernel oil, olive oil, safflower oil, sunflower seed oil, flaxseed (linseed) oil, corn oil, cottonseed oil, peanut oil, walnut oil, almond oil, grape seed oil, evening primrose oil, coconut oil, borage oil and blackcurrant oil); marine oils (e.g., fish oils and fish liver oils), or a mixture thereof.

In some embodiments, the lipid comprises oils containing medium-chain triglycerides (“MCTs”), such as coconut oil, palm kernel oil and butter or MCTs in purified form. The lipid-soluble matrix further enhances solubility of the nutraceutical.

The nutraceutical can be present in the composition in an amount of from 1% to 80% (e.g., from 5% to 80%, from 5% to 50%, from 5% to 40%, from 10 to 50%, from 10% to 40%, or from 10% to 30%) by weight. In some embodiments, more preferably the nutraceutical can be present in the composition in an amount of from 5% to 50% by weight, or most preferably from 10% to 30% by weight. In some embodiments, the nutraceutical does not exceed 25% by weight in the composition.

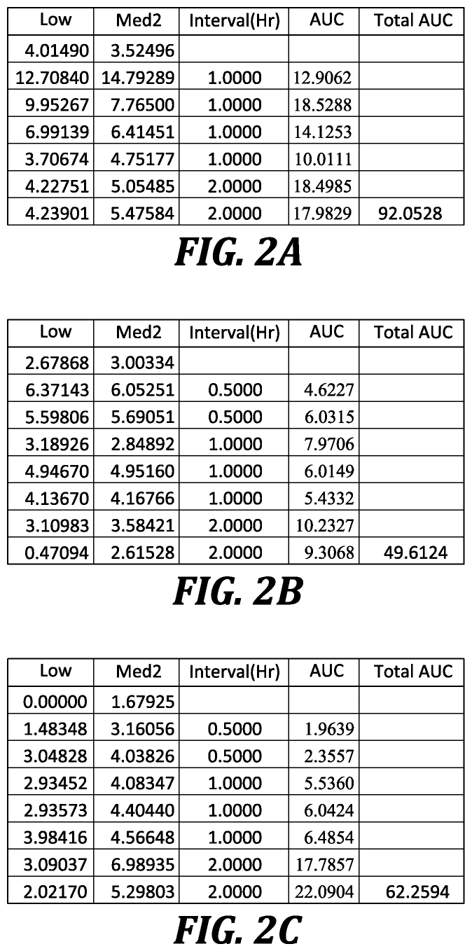

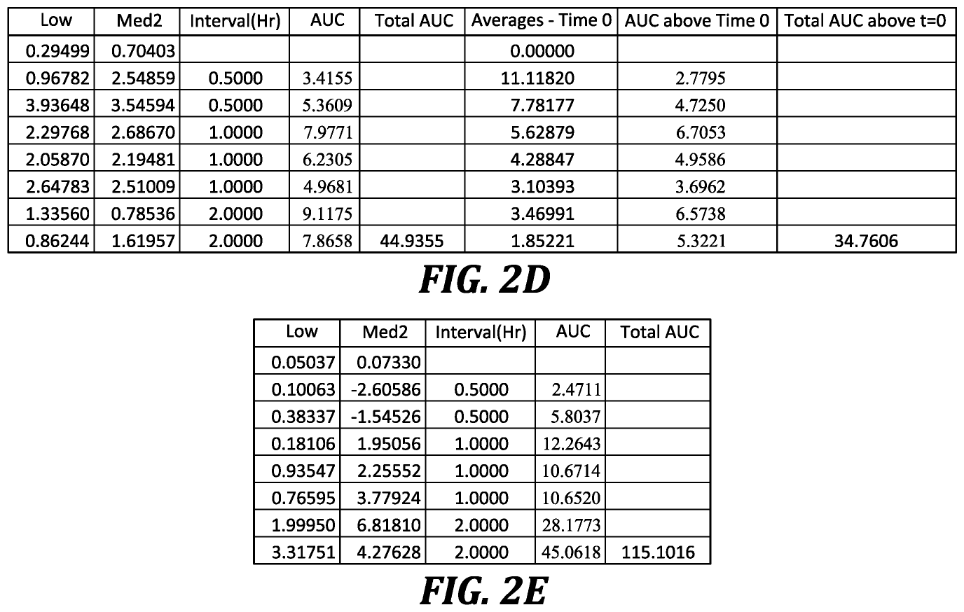

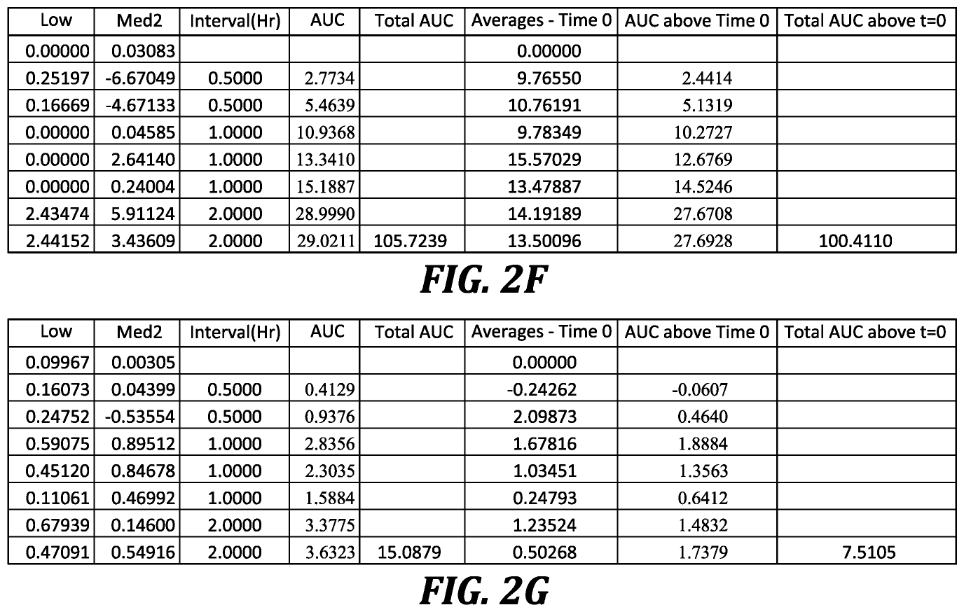

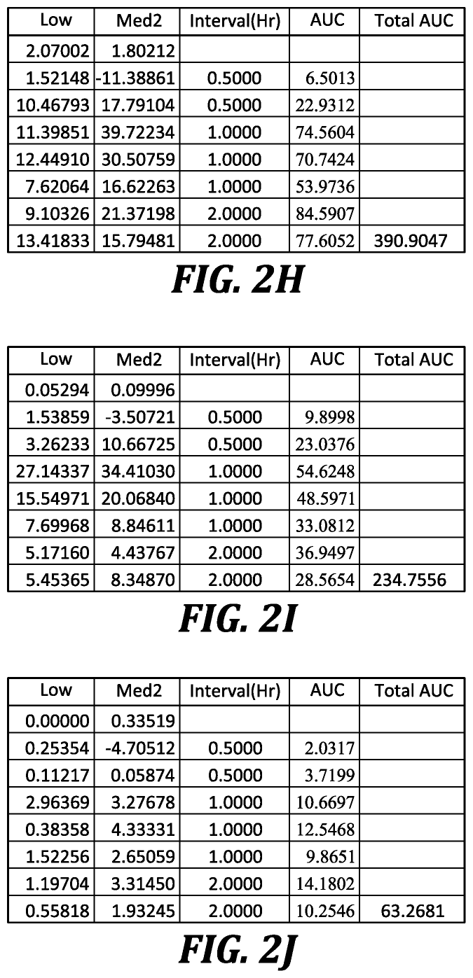

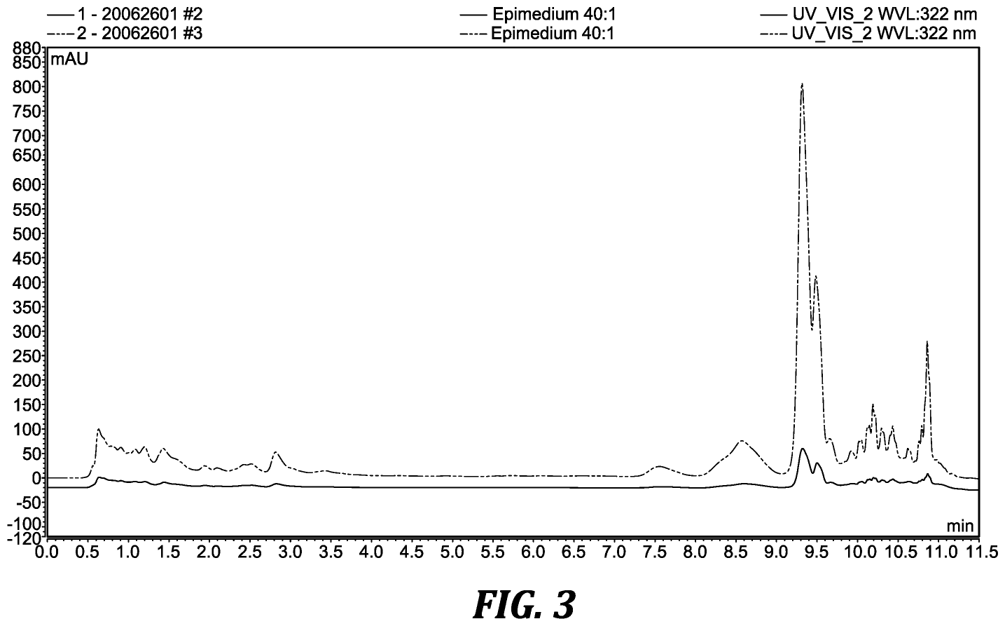

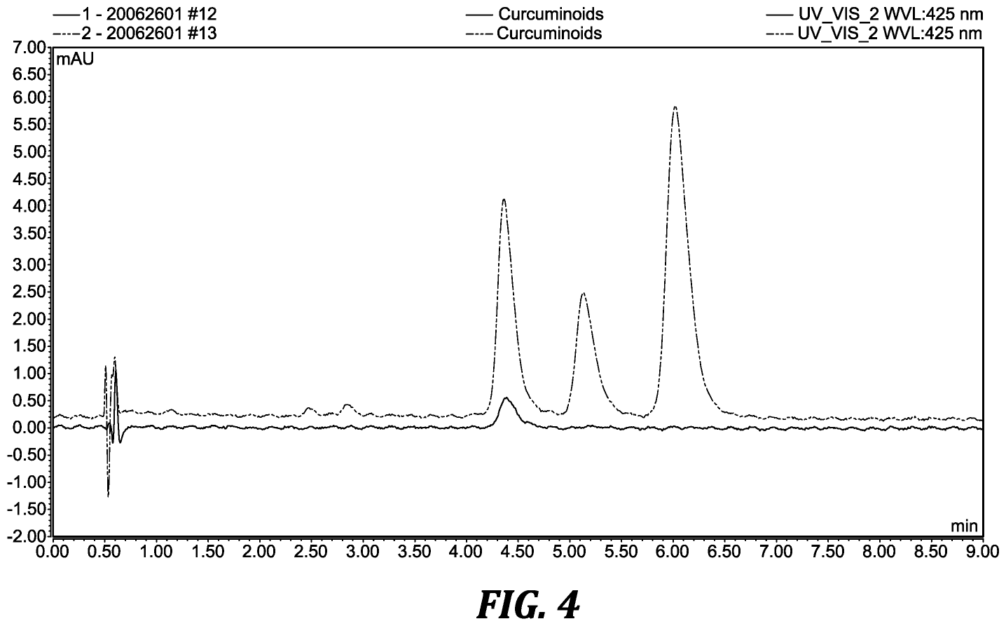

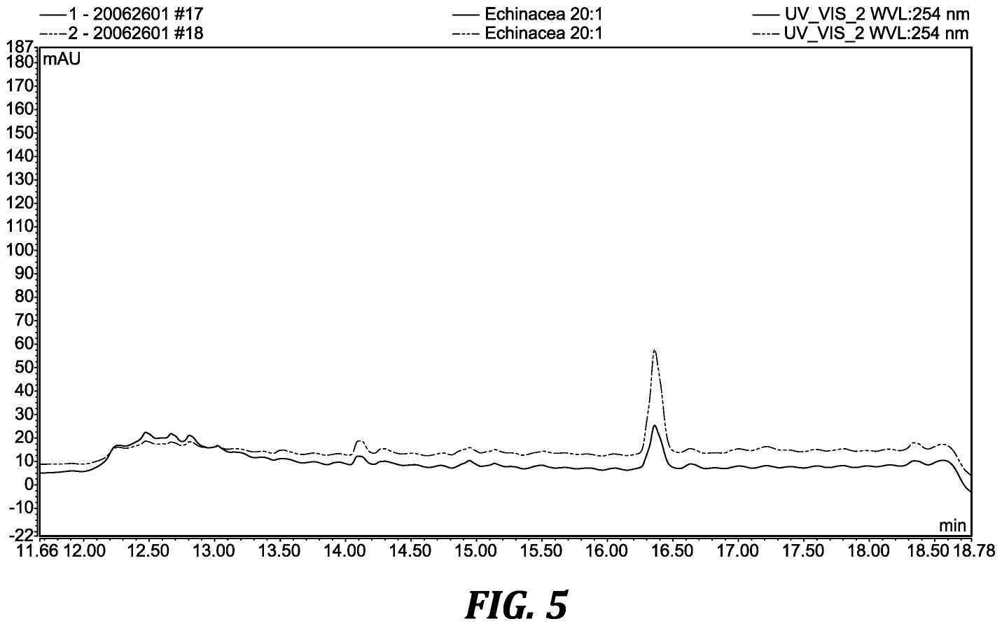

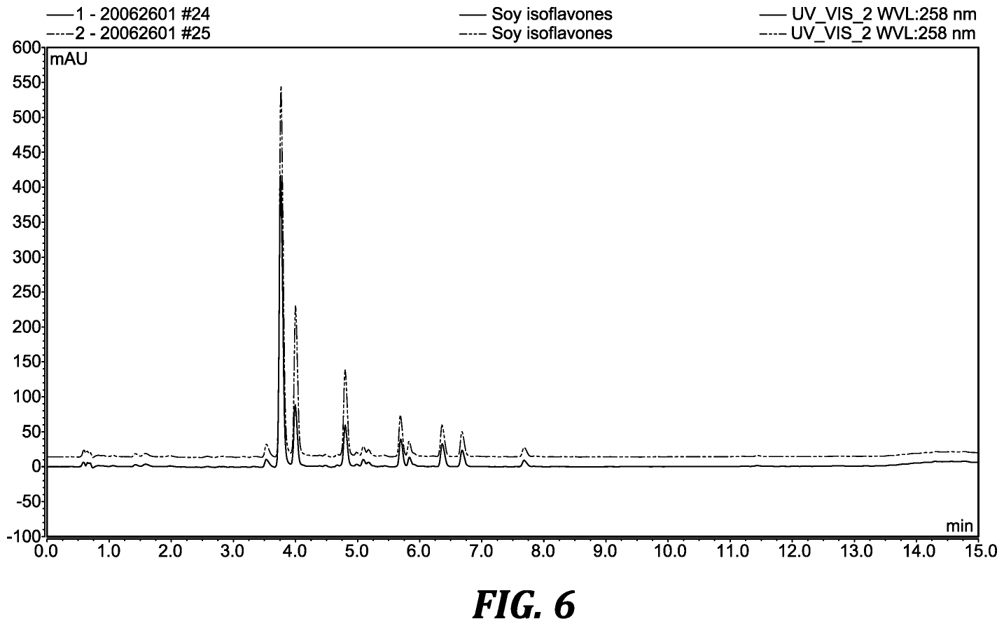

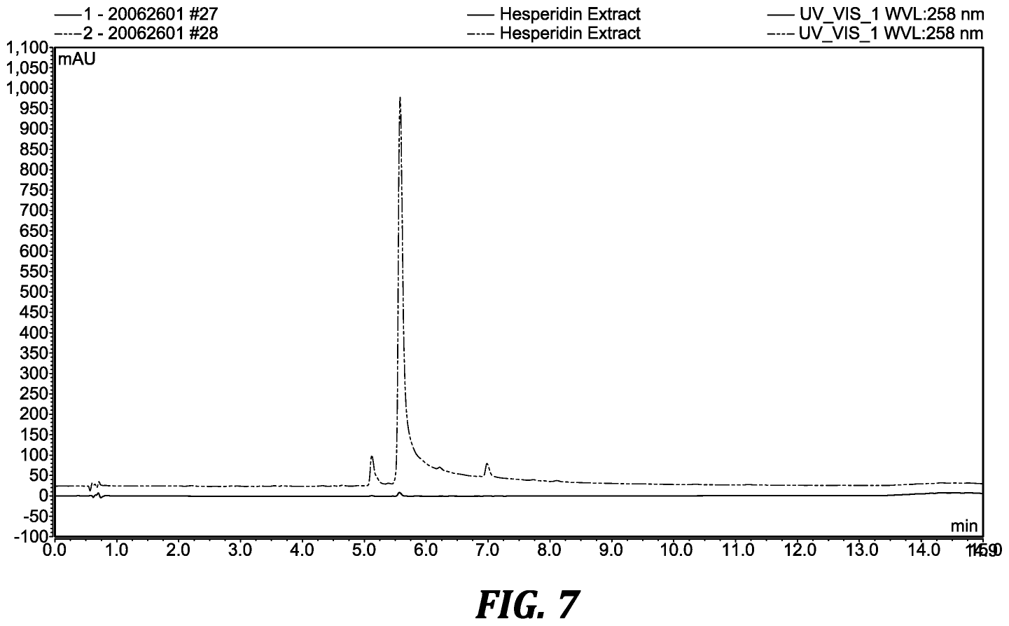

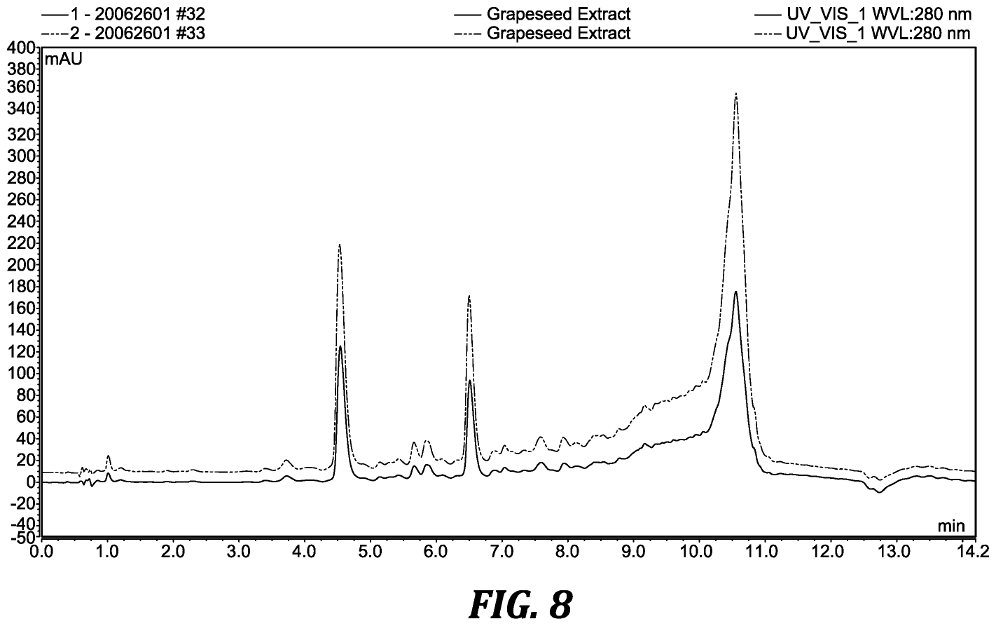

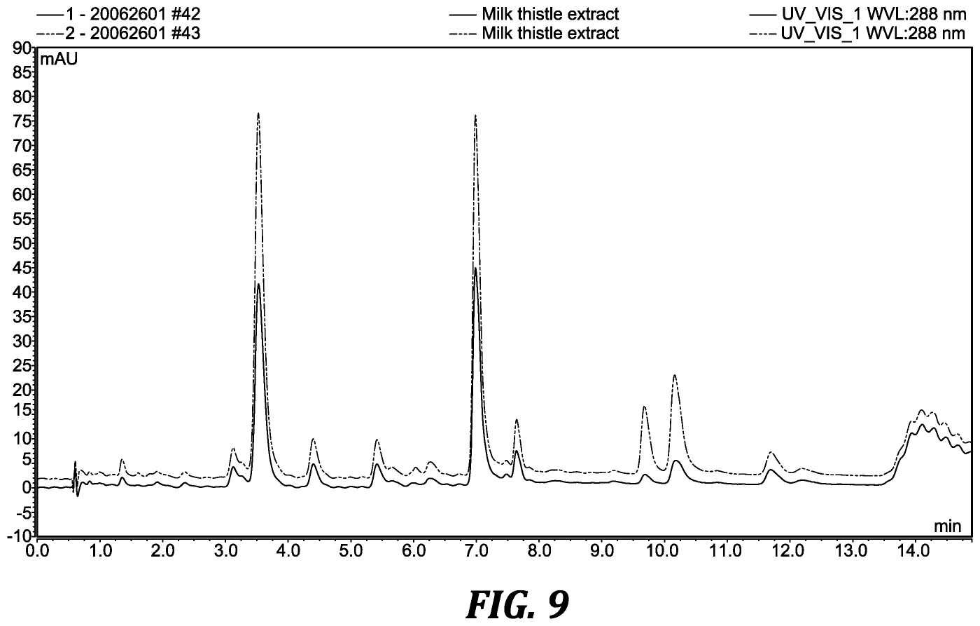

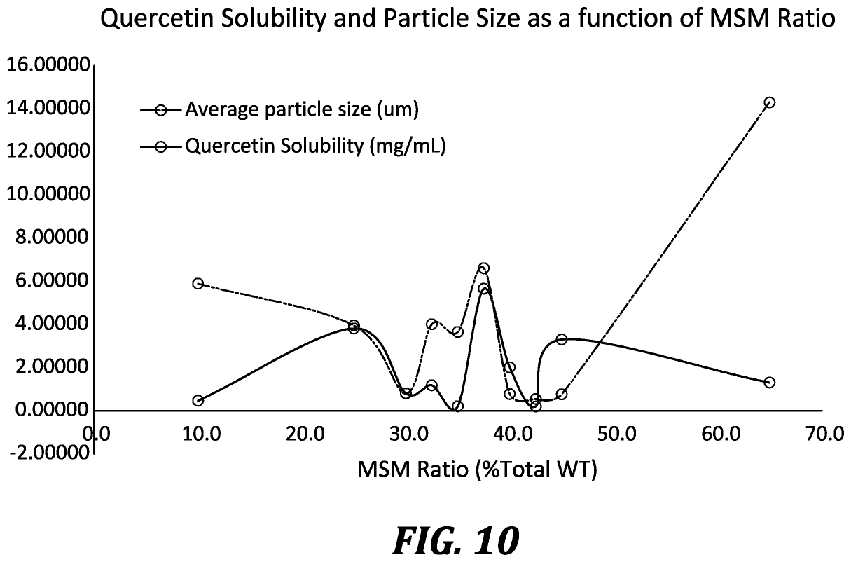

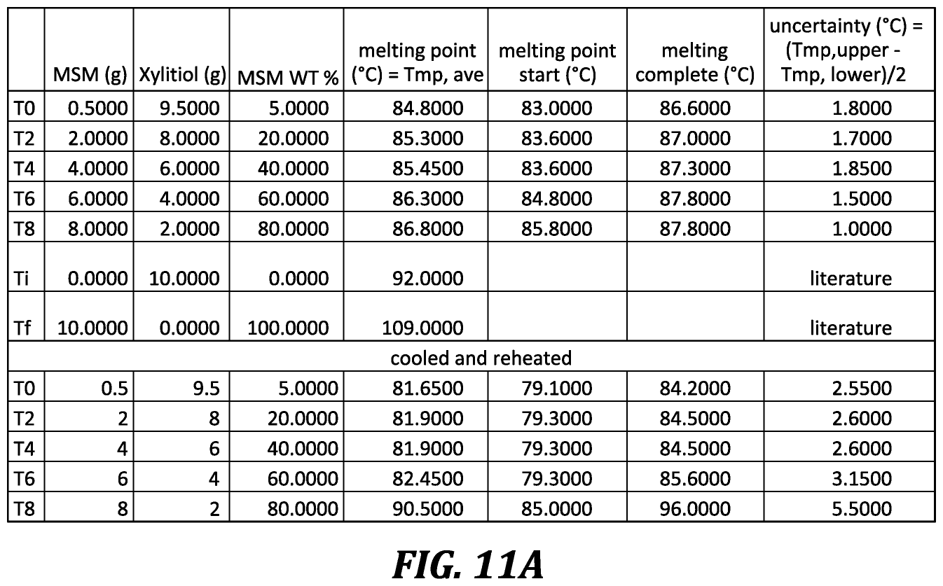

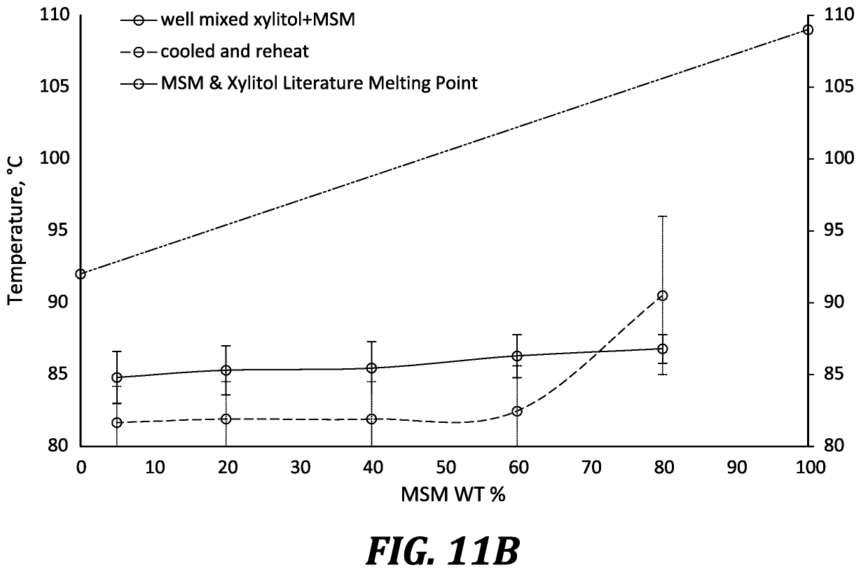

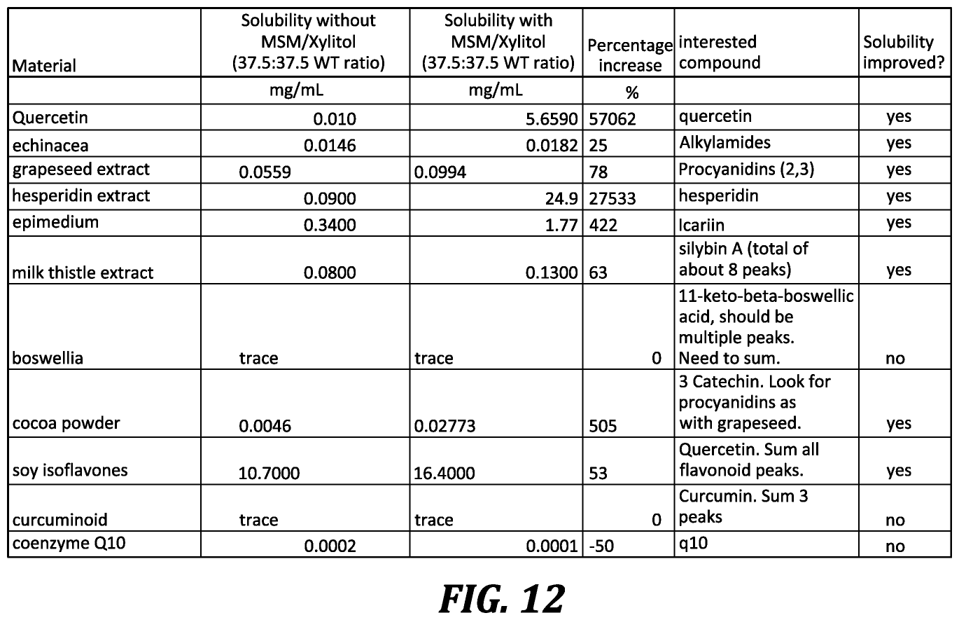

In some embodiments, when administered to a subject, the composition including the nutraceutical and the eutectic matrix can provide from 2 to 25 times (e.g., from 4 to 25 times, from 6 to 25 times, from 8 to 25 times, from 10 to 25 times, from 10 to 20 times, from 15 to 25 times, or from 15 to 20 times) the AUC_{0-8 hours} measured from the same nutraceutical without a solubilizing matrix (for example, comparing AUC from FIG. 2H with the AUC from FIG. 2G). In certain embodiments, the composition including the nutraceutical and the eutectic matrix provides an increase in AUC₀₋₈ hours of at least 100% (e.g., at least 150%, at least 200%, at least 250%, at least 300%, at least 350%, or at least 425%) when compared to the nutraceutical without a solubilizing matrix. In a preferred embodiment, the composition including the nutraceutical and the eutectic matrix provides an increase in AUC_{0-8 hours} of at least 425% when compared to the nutraceutical without a solubilizing matrix (for example, AUC from FIG. 2H compared with AUC from FIG. 2A). In some embodiments, the composition can provide the nutraceutical at a peak plasma level (Cₘₐₓ) that is higher than (e.g., up to 26 times higher than) the nutraceutical without a solubilizing matrix when administered to a subject (for example, dividing Cₘₐₓ from FIG. 2H by Cₘₐₓ from FIG. 2G).

The nutraceutical has a water solubility at least 4% by weight (at least 8% by weight, at least 10% by weight, or at least 20% by weight) greater compared to the water solubility of the nutraceutical in each individual matrix component (e.g., MSM, or a sugar alcohol). Without wishing to be bound by theory, it is believed that water solubility is correlated with bioavailability, and that greater water solubility provides greater bioavailability. In some embodiments, the nutraceutical has a water solubility that is not linearly correlated to a diameter of the particles. In certain embodiments, the nutraceutical has a water solubility that is not linearly correlated with a percentage of methylsulfonylmethane in the composition.

In some embodiments, the nutraceutical has a peak water solubility at a methylsulfonylmethane concentration of from 20% to 50% by weight of the composition and the sugar alcohol concentration of 25% to 55% by weight of the composition. In some embodiments, the nutraceutical in the eutectic matrix has a water solubility greater than the sum of the water solubilities of the nutraceutical in each of components of the eutectic matrix (i.e., in each of the methylsulfonylmethane and sugar alcohol(s)).

The composition can be in the form of a tablet, a pill, a capsule, a softgel, a gummy, a powder, or a liquid. The tablets or pills can be coated. In some embodiments, the tablets or pills are coated with an enteric layer or coating. A variety of materials can be used for the enteric layers or coatings, such materials including a number of polymeric acids and mixtures of polymeric acids with such materials as shellac, cetyl alcohol, and cellulose acetate. The liquid composition can be made by first formulating the composition in a powder, then dissolving or suspending the powder in a liquid vehicle.

### Methods of Making the Compositions

Methods of Making the Eutectic Matrix

The eutectic matrices of the present disclosure can be made, for example, by mechanically mixing MSM and a sugar alcohol together. Then the mixture can be heated at a temperature where the components of the eutectic matrix are completely melted. Depending on the sugar alcohol, the heating temperature can be from 70° C. to 125° C.

In some embodiments, one component of the eutectic mixture can be melted first at its melting point or at a temperature higher than its melting point, and then the other component(s) can be dissolved in the melted component. The eutectic matrix solidifies upon cooling.

In some embodiments, the mixture of MSM and a sugar alcohol is dissolved in water, ethanol, acetone, or a suitable polar solvent, or in a combination of polar solvents. Or MSM and the sugar alcohol could be separately dissolved in the same or different polar solvents, and then combined together. The eutectic matrix is then re-crystalized from the solution or the solution is evaporated through spray-drying, vacuum-drying, freeze-drying, or other drying techniques to obtain the eutectic matrix in solid form.

Methods of Making the Nutraceutical Composition

In some embodiments, the nutraceutical is added directly to the liquefied eutectic matrix in one of the processes described above, with or without additional solvents, prior to obtaining the eutectic matrix in solid form. The composition can be obtained when the eutectic matrix solidifies, or when solvents are evaporated to obtain the eutectic matrix in solid form.

While methods of making the eutectic matrix and the nutraceutical composition are described above and in the Examples below, it is understood that the methods can be readily adapted and modified as known by a person of skill in the art.

### Methods of Characterizing the Compositions

Bioavailability

Between 4 to 8 volunteer subjects aged between 25 and 55 years of age can be selected as test subjects to compare the various nutraceutical compositions of the present disclosure. After an overnight fast, blood samples can be collected from each volunteer before ingesting the composition being tested. Blood can be again collected at 0.5 hours, 1 hour, 2 hours, 3 hours, 4 hours, 6 hours, and 8 hours. After all blood samples are collected, the blood samples can be processed as follows.

1. Pipette a fix amount of blood (e.g., 50-100 μL) into a microcentrifuge vial;

2. Add sufficient amount of enzyme (e.g., 100 μL) into the vial;

3. Incubate the vial in the 37° C. for 1 hour to hydrolyze the nutraceutical conjugates;

4. Add 10 μl of an Internal Standard (IS) working solution and 400 μl of a suitable solvent (typically ethanol, methanol, acetone, or isopropanol) to the vial;

5. Sonicate the mixture for 10 minutes to 1.5 hours at 25°−50° C. to extract the analytes;

6. Centrifuge at 3000 g or higher for 5 minutes and then place the supernatant in a suitable container for subsequent analysis,

7. Run liquid chromatography mass spectrometry (LC-MS) analysis and analyze for blood concentrations of the nutraceutical. AUC values are calculated by integrating the area under the curve for the average blood concentrations over time for the product being tested.

Water Solubility

25-500 mg of a composition containing the equivalent of about 25 mg of the nutraceutical can be added to 10 mL of distilled water in a screw cap test tube. The test tube can be vortexed for 2 minutes and sonicated in a water bath set to the specified temperature for 1 hour. Then samples can be filtered 0.45 μm syringe filter and then analyzed by high-pressure liquid chromatography (HPLC). The filtrate after the filtration step contains water-soluble particles smaller than 450 nm. If the concentration determined by HPLC is not within the calibration curve, the sample can further diluted to a suitable concentration and re-analyzed.

The following examples are included for the purpose of illustrating, not limiting, the described embodiments.

### EXAMPLES

### Example 1. Quercetin Bioavailability Assessment

Participants consumed standardized breakfast and lunch: Breakfast: egg and ham McMuffin with cheese, or sausage and egg McMuffin, Lunch: fried chicken (2-3 pieces), French fries, gravy, coleslaw; or beef noodle soup.

Treatments

Treatment A: Quercetin powder untreated, 500 mg divided in 3 capsules

Treatment B: Softgel fill formulation with ethanol, 500 mg divided in 3 capsules

Treatment C: Granulated quercetin (40-60 mesh filled in hard gel capsules)

Treatment D: Peppermint oil formula (blended, filled in hard gel capsules)

Treatment E: Naka Extra strength quercetin 500 mg-1 capsule

Treatment F: New Roots Herbal quercetin bioflavonoids 500 mg-1 capsule

Treatment G: Amazing Nutrition Amazing formulas quercetin 500 MG-1 capsule

Treatment H: Quercetin, rutin in MSM+xylitol, 3 g dose

Treatment I: Quercetin EMIQ capsules, 10 capsules per person

Treatment J: SunActive Quercetin (cyclodextran)

Treatment K: Quercetin, rutin in MSM+xylitol, 2 g dose

Treatment L: MSM xylitol formula with MCT, 2 g (660 mg quercetin) dose

Treatment M: Quercetin EMIQ capsules, 2 capsules per person

| | | | | | | | | | P2 (starting |
|---|---|---|---|---|---|---|---|---|---|
| Participant | P1 | P2 | P3 | P4 | P5 | P6 | P7 | P8 | treatment B) |
| Treatment A | Yes | Yes | Yes | Yes | Yes | Yes | Yes | Yes | Yes |
| Treatment B | Yes | No | Yes | Yes | Yes | Yes | Yes | Yes | Yes |
| Granulated | Yes | No | Yes | Yes | Yes | Yes | Yes | Yes | Yes |
| quercetin (powder) | | | | | | | | | |
| Treatment C | No | No | No | No | No | No | Yes | No | No |
| Treatment C | Yes | No | Yes | Yes | Yes | Yes | No | Yes | Yes |
| Treatment D | Yes | No | Yes | Yes | Yes | Yes | No | Yes | Yes |
| Treatment E | Yes | No | Yes | Yes | Yes | No | Yes | Yes | Yes |
| Treatment F | Yes | No | Yes | Yes | Yes | Yes | Yes | Yes | Yes |
| Treatment G | Yes | No | Yes | Yes | Yes | Yes | Yes | Yes | Yes |
| Treatment H | Yes | No | Yes | Yes | Yes | Yes | Yes | Yes | Yes |
| Treatment I | Yes | No | Yes | Yes | Yes | Yes | Yes | Yes | Yes |
| Treatment J | Yes | No | Yes | No | Yes | Yes | Yes | No | Yes |
| Treatment K | No | No | Yes | Yes | No | No | Yes | No | Yes |

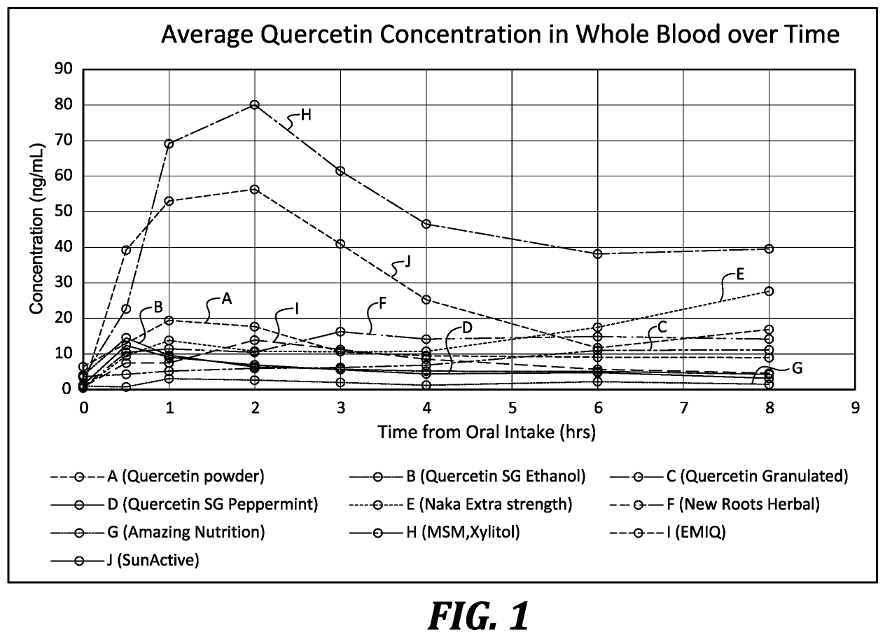

FIG. 1 shows that when subjects were administered Treatment H, a composition containing the eutectic matrix, quercetin reached higher blood concentrations compared to all other treatments. Treatment H also provided higher bioavailability as measured through AUC_{0-8 hours} compared to other treatments. FIGS. 2A to 2J contained data used to create FIG. 1. AUC was calculated numerically so that comparison could be made easily. Cₘₐₓ could be determined from these tables by looking at Column 3.

### Example 2. Water Solubility Measurements

TABLE 1
Solubility measurements

| Injection | | Solubility in Water (mg/mL) | | | |
|---|---|---|---|---|---|
| Name | Comment | Rutin | Isoquercitrin | Quercetin | Total |
| Quercetin | 5.0 g quercetin mixed | 0 | 0 | 0 | 0 |
| solubility | in 10.0 g melted | | | | |
| MSM | MSM. Stirred in | | | | |
| | water, then filtered | | | | |
| | through 0.45 μm | | | | |
| | PTFE filter. | | | | |
| Quercetin | 5.0 g quercetin 10.0 g | 0.0009 | 0 | 0 | 0.0009 |
| solubility | MSM, 1.0 g H₂O. | | | | |
| MSM + | Heated and stirred | | | | |
| choline | until granules formed | | | | |
| | without melting | | | | |
| | MSM. Stirred in | | | | |
| | water then filtered | | | | |
| | through 0.45 μm | | | | |
| | PTFE filter. | | | | |
| Quercetin | Approx. equal amount | 0 | 0 | 0.0044 | 0.0044 |
| solubility Vit | of vitamin C and | | | | |
| C, choline, | choline Cl dissolved | | | | |
| Water | in minimal water and | | | | |
| | then heated until thick | | | | |
| | liquid remains. Add | | | | |
| | Quercetin and cool | | | | |
| | into paste. Stirred in | | | | |
| | water and filtered | | | | |
| | through 0.45 μm | | | | |
| | PTFE filter. | | | | |
| Quercetin | 5.0 g quercetin 10.0 g | 0 | 0 | 0 | 0 |
| solubility | MSM, 1.0 g H₂O. | | | | |
| MSM + H₂O | Mixed with minimal | | | | |
| | heating. Material | | | | |
| | remains as fine | | | | |
| | powder. Stirred in | | | | |
| | water then filtered | | | | |
| | through 0.45 μm | | | | |
| | PTFE filter. | | | | |
| Quercetin | 5.0 g quercetin 10.0 g | 0 | 0 | 0 | 0 |
| solubility | MSM, 1.0 g H₂O. | | | | |
| MSM + H₂O + | Heated and stirred | | | | |
| heat | until granules formed | | | | |
| | without melting | | | | |
| | MSM. Stirred in | | | | |
| | water then filtered | | | | |
| | through 0.45 μm | | | | |
| | PTFE filter. | | | | |
| Quercetin | 5.0 g quercetin mixed | 0 | 0 | 0.0057 | 0.0057 |
| solubility | in 10.0 g xylitol. | | | | |
| xylitol | Heated until xylitol is | | | | |
| | melted. Stirred in | | | | |
| | water, then filtered | | | | |
| | through 0.45 μm | | | | |
| | PTFE filter. Solution | | | | |
| | murky after filtering. | | | | |
| Quercetin | 5.0 g quercetin + 5.0 g | 0 | 0 | 0.0758 | 0.0758 |
| solubility | xylitol + 5.0 g MSM. | | | | |
| xylitol MSM | Heated until melted. | | | | |
| | Stirred in water, then | | | | |
| | filtered through 0.45 | | | | |
| | μm PTFE filter. | | | | |
| | Solution murky after | | | | |
| | filtering. | | | | |
| Quercetin | 5.0 g quercetin + 5.0 g | 0.001 | 0 | 0.0705 | 0.0715 |
| solubility | sorbitol + 5.0 g MSM. | | | | |
| sorbitol MSM | Heated until melted. | | | | |
| | Stirred in water, then | | | | |
| | filtered through 0.45 | | | | |
| | μm PTFE filter. | | | | |
| | Solution clear after | | | | |
| | filtering. | | | | |
| MSM choline | 5.0 g quercetin + 5.0 g | 0.0019 | 0 | 0.0093 | 0.0112 |
| bitartrate | choline bitartrate + | | | | |
| quercetin | 5.0 g MSM. Heated | | | | |
| | until melted. Stirred | | | | |
| | in water, then filtered | | | | |
| | through 0.45 μm | | | | |
| | PTFE filter. Solution | | | | |
| | clear after filtering. | | | | |
| MSM xylitol | 5.0 g rutin + 5.0 g | 0.877 | 0.0211 | 0.0869 | 0.985 |
| rutin | xylitol + 5.0 g MSM. | | | | |
| | Heated until melted. | | | | |
| | Stirred in water, then | | | | |
| | filtered through 0.45 | | | | |
| | μm PTFE filter. | | | | |
| | Solution murky after | | | | |
| | filtering. | | | | |
| MSM xylitol | 2.5 g rutin + 2.5 g | 0.4483 | 0.0029 | 0.4357 | 0.8869 |
| rutin quercetin | quercetin + 5.0 g | | | | |
| | xylitol + 5.0 g MSM. | | | | |
| | Heated until melted. | | | | |
| | Stirred in water, then | | | | |
| | filtered through 0.45 | | | | |
| | μm PTFE filter. | | | | |
| | Solution murky after | | | | |
| | filtering. | | | | |
| MSM xylitol | 2.5 g rutin + 2.5 g | 0.2605 | 0.003 | 0.4065 | 0.67 |
| rutin | quercetin + 5.0 g | | | | |
| quercetin#2 | xylitol + 5.0 g MSM. | | | | |
| | Heated until melted. | | | | |
| | Stirred in water, then | | | | |
| | filtered through 0.45 | | | | |
| | μm PTFE filter. | | | | |
| | Solution murky after | | | | |
| | filtering. | | | | |

The water solubilities of the various compositions were tested by HPLC to obtain mg dissolved component/milliliter water solubility. One of the combinations is MSM with choline. While choline is commonly used in other known eutectic systems, solubility increases much less with choline compared to a sugar alcohol, such as xylitol. This demonstrates the unexpected effectiveness of MSM with a sugar alcohol (e.g., xylitol) in improving solubility.

Other nutraceuticals were also tested for eutectic mixtures with other components but did not work as well as MSM with sugar alcohol (e.g., xylitol). Vitamin C with choline and water is a known eutectic system, but it could not solubilize quercetin as well as MSM with xylitol. Other combinations that were tested are MSM with choline, MSM by itself, xylitol by itself, and MSM with sorbitol. Without wishing to be bound by theory, it is believed that AUC is correlated with dosage. FIG. 1 demonstrates that the MSM-sugar alcohol eutectic matrix uniquely increases bioavailability because all of the treatments administered contained the same dosage of quercetin.

### Example 3. Non-Linear Solubility Behavior in MSM: Xylitol Matrix

25-500 mg of the composition containing the equivalent of about 25 mg of the active ingredient was added to 10 mL of distilled water in a screw cap test tube. The test tube was vortexed for 2 minutes and sonicated in a water bath set to the specified temperature for 1 hour. Then samples were filtered 0.45 μm syringe filter and then analyzed by HPLC. The filtrate after the filtration step contained only water-soluble particles smaller than 450 nm in dimension. If the concentration determined by HPLC was not within the calibration curve, the sample was further diluted to a suitable concentration and re-analyzed.

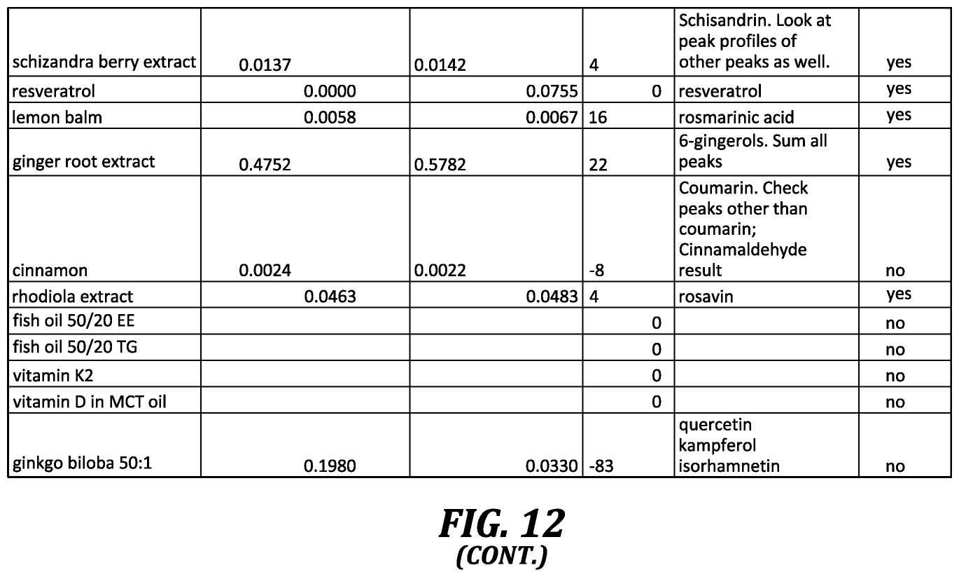

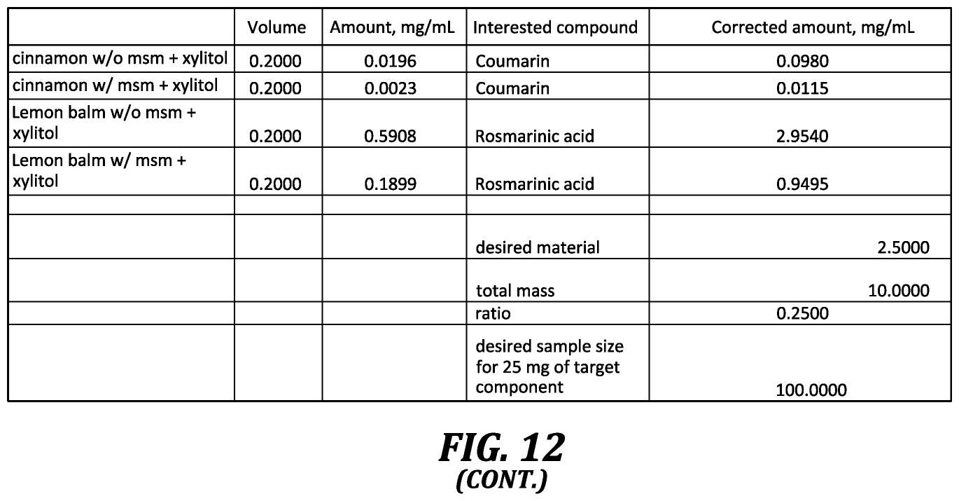

FIG. 12 demonstrates that many poorly soluble nutraceuticals have improved solubility when incorporated into an MSM-xylitol eutectic matrix.

### Example 4. Solubility Studies

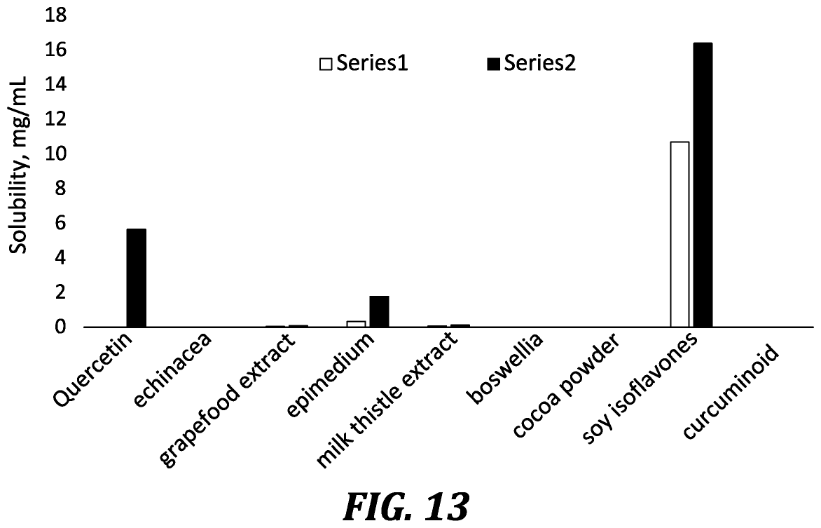

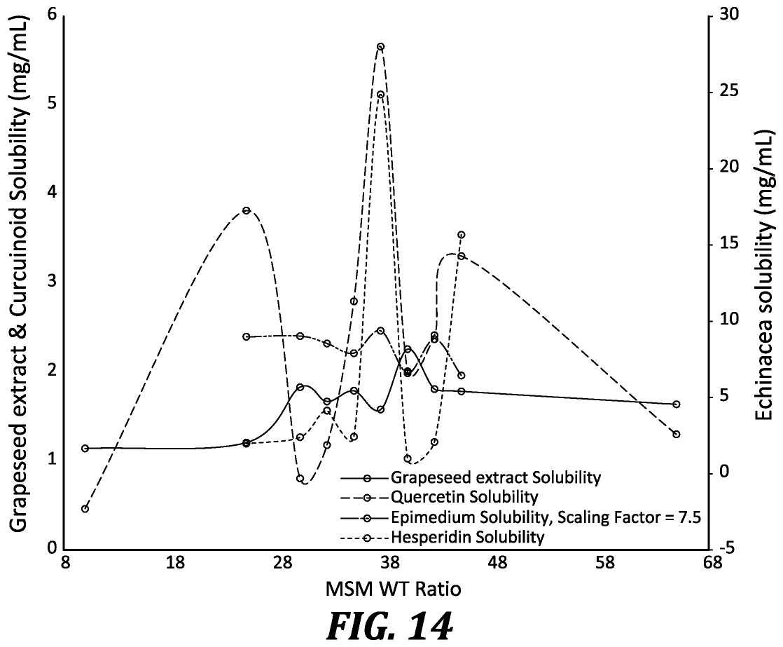

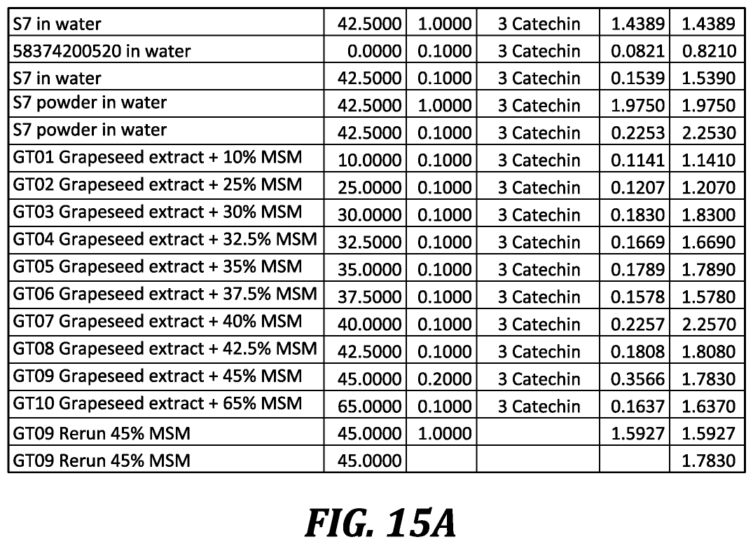

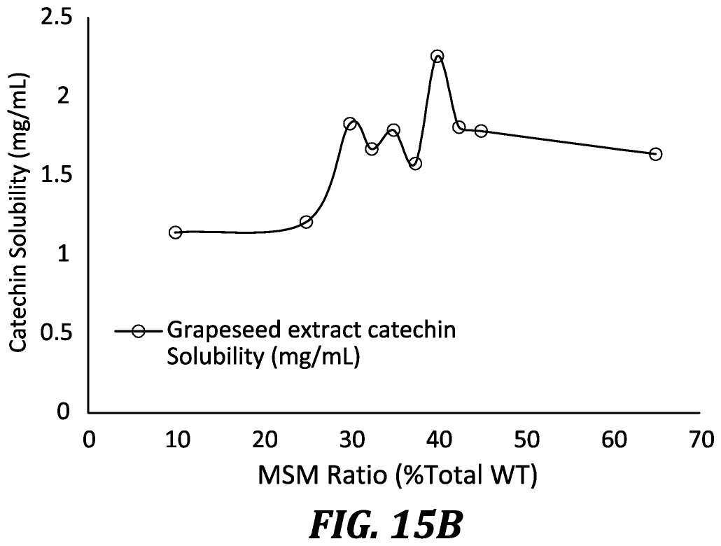

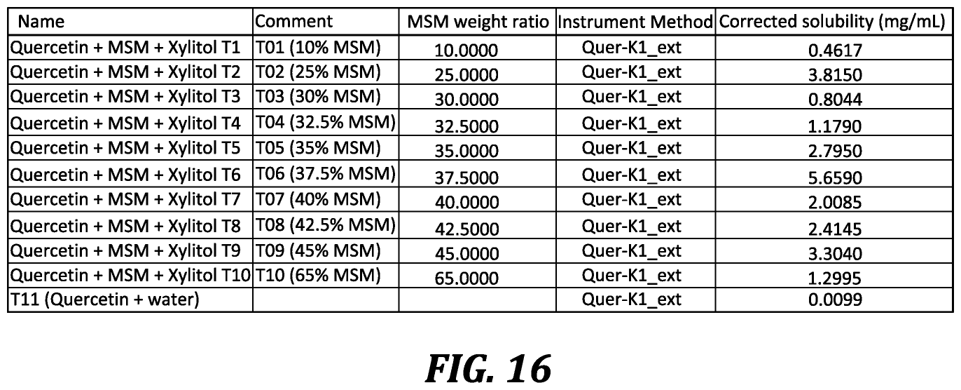

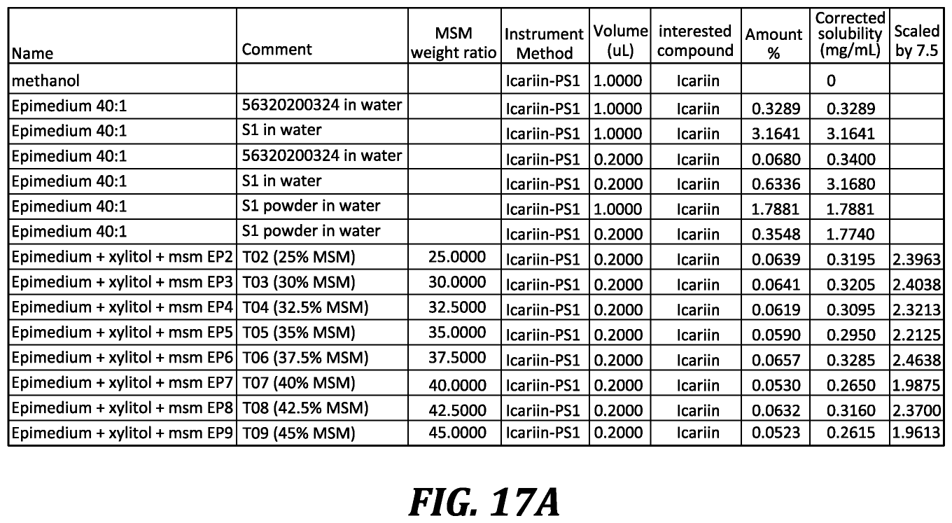

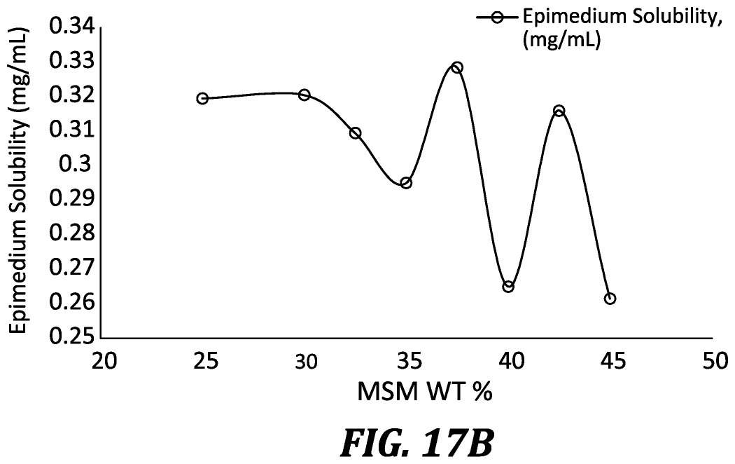

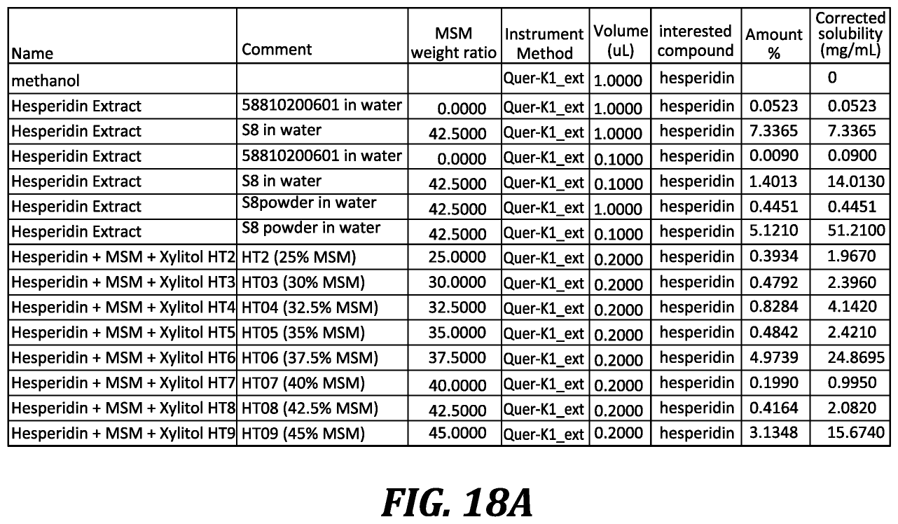

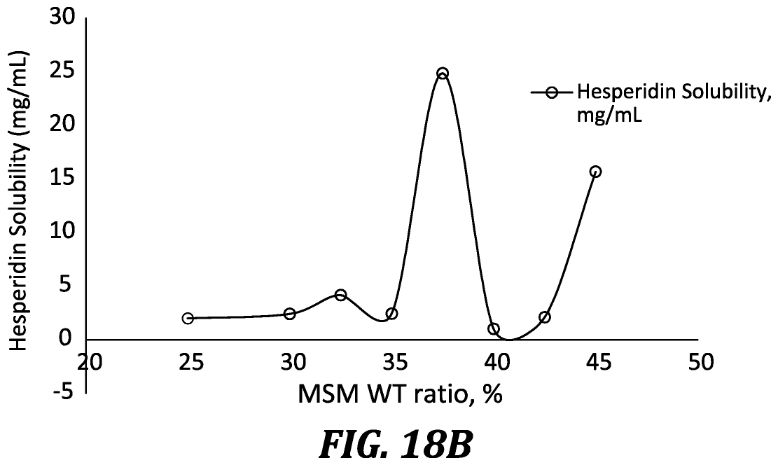

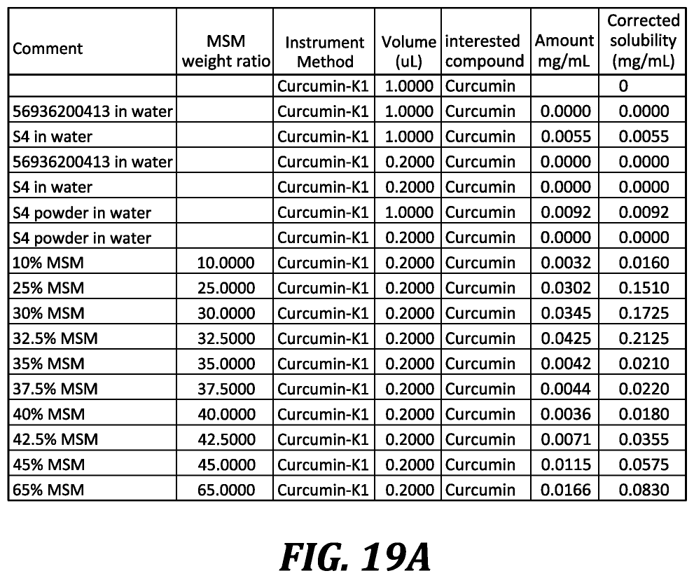

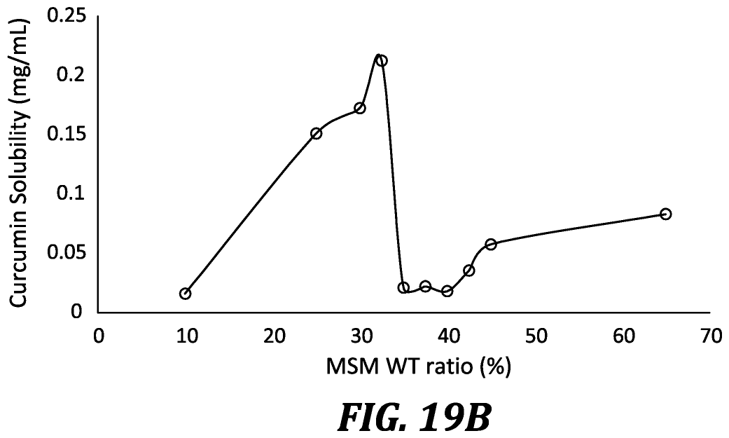

FIG. 13 shows that solubility in water improved for many nutraceutical components in a composition of the present disclosure. FIG. 14 compares the solubility as a function of MSM ratio in an MSM-sugar alcohol eutectic matrix and demonstrates that the optimal ratio occurred around 38% (corresponding to 50:50 MSM:Xylitol). FIGS. 15A, 15B, 16, 17A, 17B, 18A, 18B, to 19 show the raw data for FIG. 14.

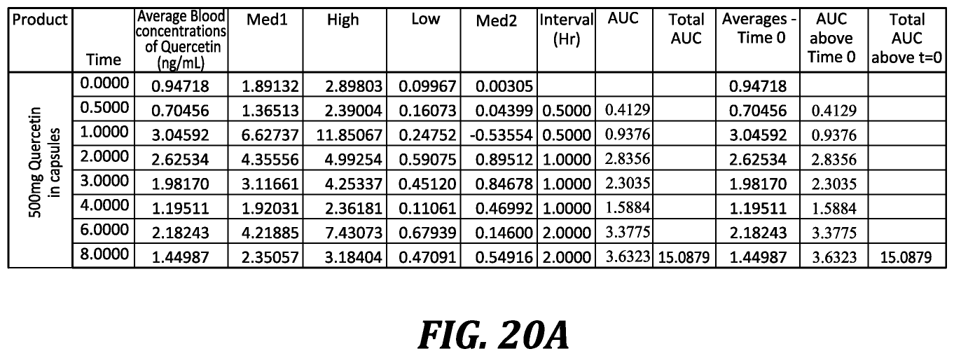

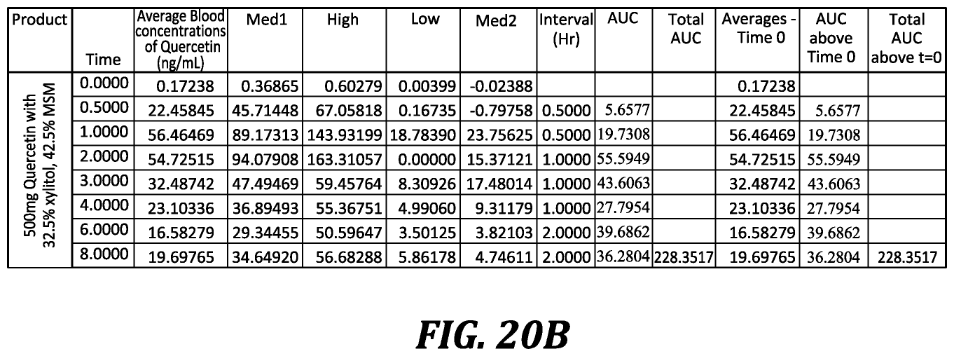

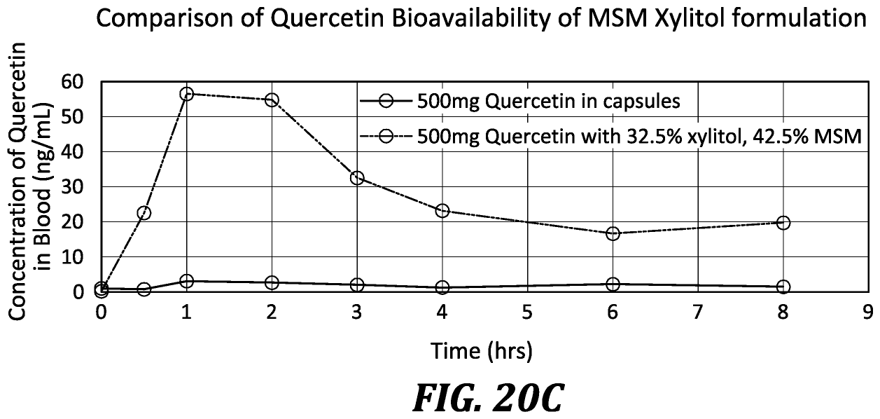

FIGS. 20A-20C show the bioavailability comparison of quercetin with 42.5% MSM compared to quercetin without a solubilization matrix. When compared against the data in FIG. 14, the ratio of MSM to xylitol in this treatment was at a point where solubility in water was at a minimum. Yet, this treatment still showed unexpectedly high bioavailability when administered. This demonstrated that the increase in bioavailability of the MSM-sugar alcohol eutectic matrix could not be explained by an increase in water solubility alone.

### Example 5. Lipid-Soluble Matrix Studies

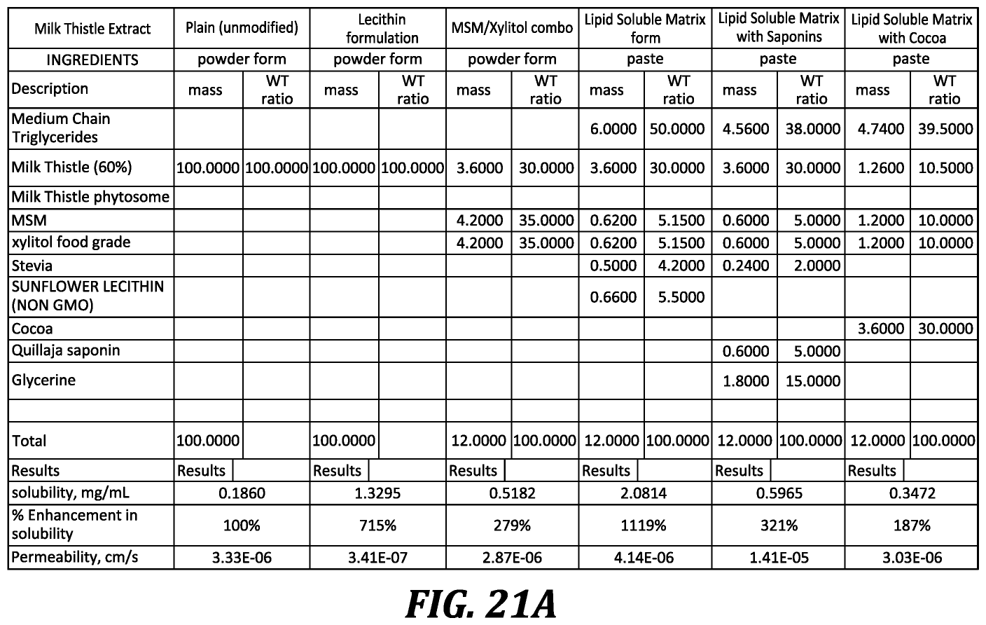

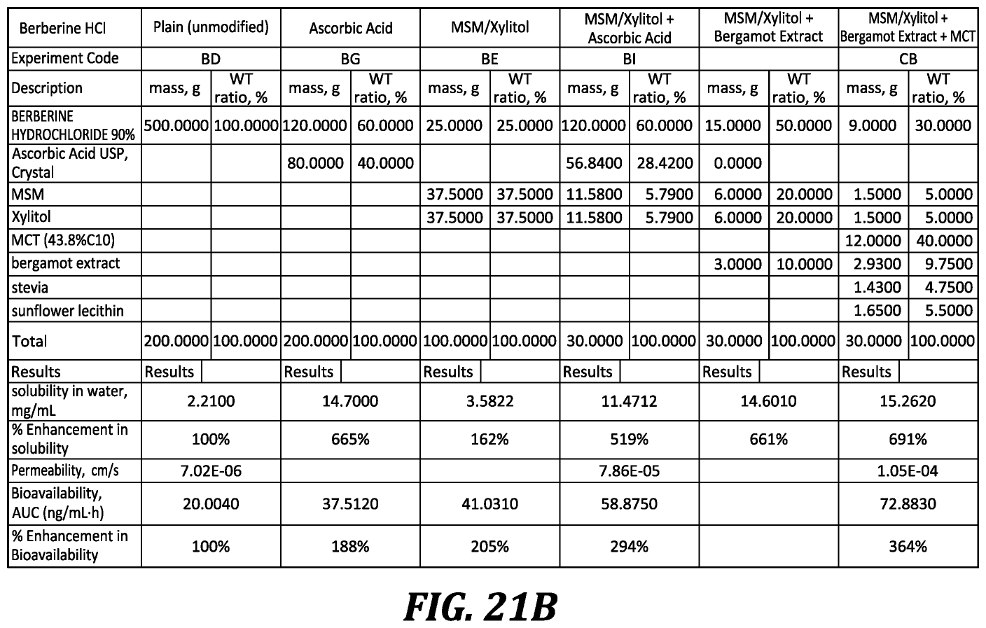

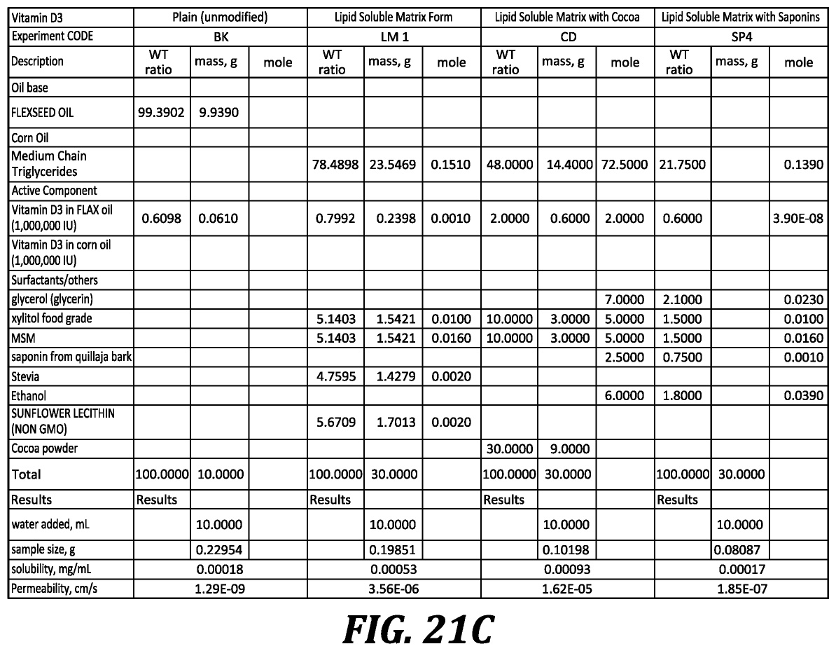

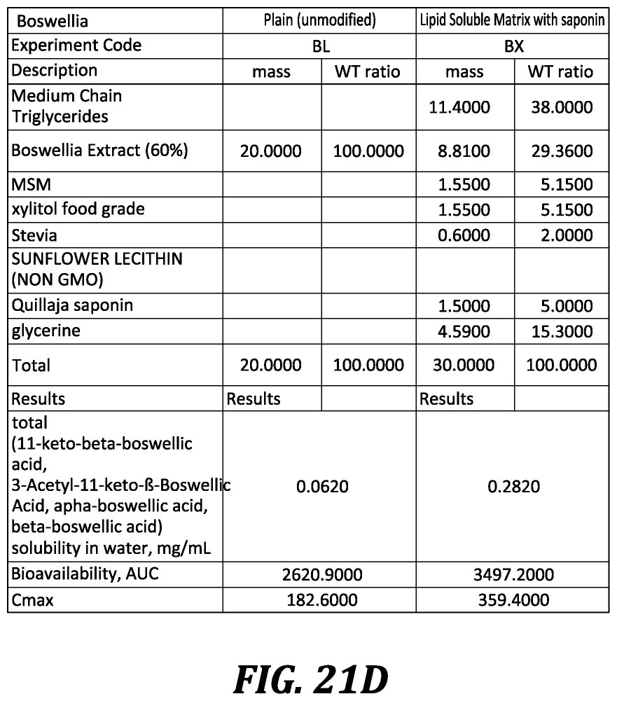

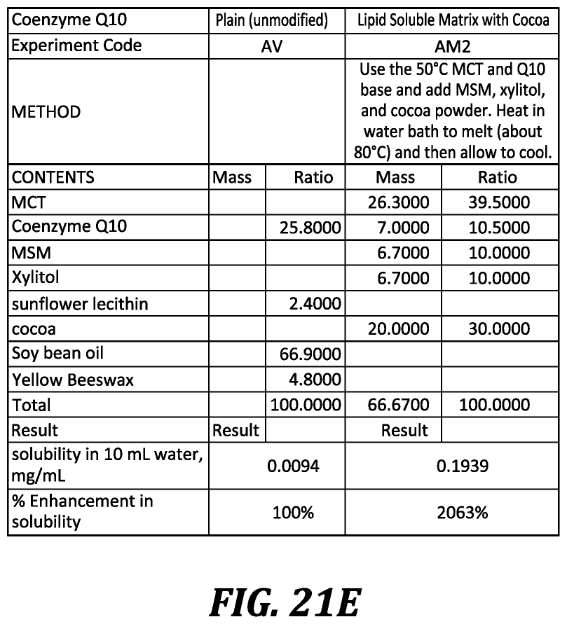

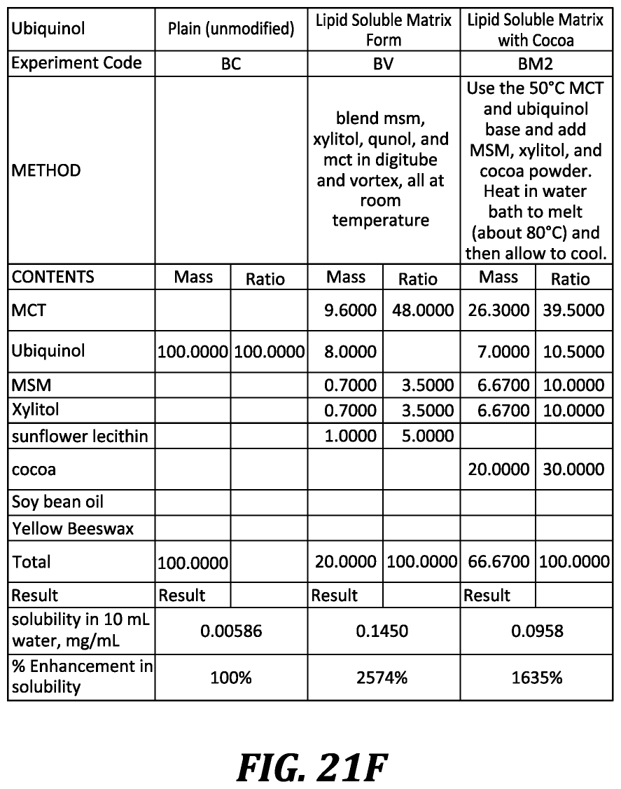

A eutectic matrix comprising methylsulfonlmethane and a sugar alcohol can be combined with an oil to form a lipid-soluble matrix that further enhances the solubility of the nutraceutical. FIG. 21A shows that a eutectic matrix comprising methylsulfonlmethane and a sugar alcohol alone (MSM/xylitol) can enhance the solubility of a plain, unmodified form of milk thistle extract by 279%. When the eutectic matrix and milk thistle extract are combined with an oil, for example MCTs, the solubility of the milk thistle extract is increased to 1119%. Combining the lipid-soluble matrix with saponins or cocoa further increases the solubility of the lipid-soluble matrix form of milk thistle extract compared to the unmodified form of milk thistle extract, but not to the same extent as the lipid-soluble matrix without saponins or cocoa.

FIG. 21B shows that a eutectic matrix comprising methylsulfonlmethane and a sugar alcohol can enhance bioavailability independent of solubility. As shown in FIG. 21B, the addition of ascorbic acid to berberine extract increases the solubility of the berberine extract by 665%, which is more than the 162% increase in solubility of berberine extract in the MSM/xylitol form, and more than the 519% increase in solubility of berberine extract in the MSM/xylitol with ascorbic acid form. However, the bioavailability of the MSM/xylitol with ascorbic acid form is higher than the bioavailability of the berberine extract with ascorbic acid form, even though former has lower solubility than latter.

The results further demonstrate that the addition of a secondary ingredient can increase the bioavailability provided by the eutectic matrix comprising methylsulfonylmethane and a sugar alcohol. Both ascorbic acid and bergamot extract are shown to be useful secondary ingredients to increase bioavailability.

FIG. 21C demonstrates the increased permeability of lipid-soluble vitamins and nutraceutical ingredients. As shown in FIG. 21C, the permeability of vitamin D3 through a Caco-2 cell layer is enhanced by a eutectic matrix comprising methylsulfonylmethane and a sugar alcohol combined with an oil to form a lipid-soluble matrix. The addition of cocoa further increases the permeability of vitamin D3 to 456%. The addition of saponins, on the other hand, decreases the permeability of vitamin D3 compared to the lipid-soluble matrix and to the lipid-soluble matrix with cocoa.

FIG. 21D shows that a eutectic matrix comprising methylsulfonylmethane and a sugar alcohol combined with an oil to form a lipid-soluble matrix, together with the addition of saponins, significantly increases the bioavailability of boswellia and the maximum concentration of boswellia in the blood.

FIG. 21E demonstrates that a eutectic matrix comprising methylsulfonylmethane and a sugar alcohol combined with an oil to form a lipid-soluble matrix, together with the addition of cocoa, significantly increases the solubility in water of CoQ10. Referring to FIG. 21E, MSM, xylitol, and cocoa powder were added to a base of CoQ10 and MCTs at 50° C. The mixture was heated in a water bath to about 80° C. and then allow to cool. The results show increased solubility in water of CoQ10 compared to the solubility in water of plain, unmodified CoQ10.

FIG. 21F shows that a eutectic matrix comprising methylsulfonylmethane and a sugar alcohol combined with an oil to form a lipid-soluble matrix, either with or without the addition of cocoa, demonstrates a significant increase in the solubility of ubiquinol in water. The lipid-soluble matrix form of ubiquinol was prepared by blending ubiquinol and MCTs and vortexing the mixture at room temperature. The lipid-soluble matrix with cocoa was prepared by adding MSM, xylitol, and cocoa powder to a base of ubiquinol and MCTs at 50° C. The mixture was heated in a water bath to about 80° C. and then allow to cool. The results show increased solubility in water for the lipid-soluble matrix form of ubiquinol compared to the plain, unmodified ubiquinol and to the lipid-soluble matrix with cocoa.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A composition, comprising:
a eutectic matrix comprising methylsulfonylmethane and a sugar alcohol; and
particles of a nutraceutical in the eutectic matrix.

2. The composition of claim 1, wherein the sugar alcohol:methylsulfonylmethane ratio is from 95:5 to 20:80.

3. The composition of claim 1, wherein the sugar alcohol comprises a 4-carbon sugar alcohol, a 5-carbon sugar alcohol, a 6-carbon sugar alcohol, a 12-carbon sugar alcohol, or any combination thereof.

4. The composition of claim 3, wherein the sugar alcohol is selected from erythritol, xylitol, arabitol, ribitol, mannitol, sorbitol, galactitol, fucitol, iditol, inositol, maltitol, and any combination thereof.

5. The composition of claim 4, wherein the sugar alcohol comprises xylitol.

6. The composition of claim 1, wherein the eutectic matrix has a melting point lower than each of the melting point of the methylsulfonylmethane and the melting point of the sugar alcohol.

7. The composition of claim 1, wherein the eutectic matrix consists of methylsulfonylmethane and the sugar alcohol.

8. The composition of claim 1, wherein the methylsulfonylmethane and the sugar alcohol form an organized structure or a repeating pattern.

9. The composition of claim 1, wherein the nutraceutical forms hydrogen bonds and/or ionic bonds with the eutectic matrix.

10. The composition of claim 1, wherein the nutraceutical comprises quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof.

11. The composition of claim 10, wherein the nutraceutical comprises quercetin.

12. The composition of claim 1, wherein the composition further comprises a lipid.

13. The composition of claim 12, wherein the lipid comprises an edible oil or a mixture of oils.

14. The composition of claim 13, wherein the lipid comprises medium chain triglycerides.

15. The composition of claim 12, wherein the lipid forms a lipid-soluble matrix with the methylsulfonylmethane, the sugar alcohol, the nutraceutical.

16. The composition of any one of claim 1 or claim 12, wherein the composition further comprises saponins, cocoa, or any combination thereof.

17. The composition of claim 1, wherein the composition has an AUC_{0-8 hours} of greater than 250 ng/ml of the nutraceutical when administered to a subject.

18. The composition of claim 1, wherein the composition is in the form of a tablet, a capsule, a softgel, a gummy, or a liquid.

19. The composition of claim 1, wherein the nutraceutical has a water solubility at least 4% greater compared to the water solubility of the nutraceutical in each individual matrix component.

20. The composition of claim 1, wherein the nutraceutical has a water solubility not linearly correlated to a diameter of the particles or not linearly correlated with a percentage of methylsulfonylmethane in the composition.

21. The composition of claim 1, wherein the nutraceutical has a peak water solubility at a methylsulfonylmethane concentration of from 20% to 50% by weight.

22. The composition of claim 1, wherein the nutraceutical has a water solubility greater than the sum of the water solubilities of the nutraceutical in each of methylsulfonylmethane and sugar alcohol.

23. The composition of claim 1, wherein the composition further comprises an enteric coating.

24. A method for making the composition of claim 1, comprising:
mixing methylsulfonylmethane and a sugar alcohol to form a mixture;
heating the mixture to melt the methylsulfonylmethane and a sugar alcohol;
adding a nutraceutical composition; and
cooling the mixture to form a eutectic matrix with particles of the nutraceutical in the eutectic matrix.

25. A method for making the composition of claim 1, comprising:
dissolving methylsulfonylmethane and a sugar alcohol in water, ethanol, acetone, or a polar solvent to form a solution;
adding a nutraceutical to the solution; and
re-crystallizing the solution to obtain a eutectic matrix with particles of the nutraceutical in the eutectic matrix.

26. A method for enhancing the solubility of a nutraceutical, comprising adding a nutraceutical to a lipid-soluble matrix.

27. The method of claim 26, wherein the lipid-soluble matrix comprises methylsulfonylmethane, a sugar alcohol, and a lipid.

28. The method of claim 27, wherein the methylsulfonylmethane and the sugar alcohol form a eutectic matrix.

29. The method of claim 27, wherein the sugar alcohol:methylsulfonylmethane ratio is from 95:5 to 20:80

90. The method of claim 27, wherein the sugar alcohol comprises a 4-carbon sugar alcohol, a 5-carbon sugar alcohol, a 6-carbon sugar alcohol, a 12-carbon sugar alcohol, or any combination thereof.

31. The method of claim 30, wherein the sugar alcohol is selected from erythritol, xylitol, arabitol, ribitol, mannitol, sorbitol, galactitol, fucitol, iditol, inositol, maltitol, and any combination thereof.

32. The method of claim 31, wherein the sugar alcohol comprises xylitol.

33. The method of claim 27, wherein the lipid comprises an edible oil or a mixture of oils.

34. The method of claim 33, wherein the lipid comprises medium chain triglycerides.

35. The method of claim 26, wherein the nutraceutical comprises quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof.

36. The method of claim 35, wherein the nutraceutical comprises quercetin.

37. The method of claim 27, wherein the lipid-soluble matrix further comprises saponins, cocoa, or any combination thereof.

38. A method for enhancing the bioavailability of a nutraceutical, comprising adding a nutraceutical to a lipid-soluble matrix.

39. The method of claim 38, wherein the lipid-soluble matrix comprises methylsulfonylmethane, a sugar alcohol, and a lipid.

40. The method of claim 39, wherein the methylsulfonylmethane and the sugar alcohol form a eutectic matrix.

41. The method of claim 39, wherein the sugar alcohol:methylsulfonylmethane ratio is from 95:5 to 20:80

42. The method of claim 39, wherein the sugar alcohol comprises a 4-carbon sugar alcohol, a 5-carbon sugar alcohol, a 6-carbon sugar alcohol, a 12-carbon sugar alcohol, or any combination thereof.

43. The method of claim 42, wherein the sugar alcohol is selected from erythritol, xylitol, arabitol, ribitol, mannitol, sorbitol, galactitol, fucitol, iditol, inositol, maltitol, and any combination thereof.

44. The method of claim 43, wherein the sugar alcohol comprises xylitol.

45. The method of claim 39, wherein the lipid comprises an edible oil or a mixture of oils.

46. The method of claim 45, wherein the lipid comprises medium chain triglycerides.

47. The method of claim 38, wherein the nutraceutical comprises quercetin, epimedium extract, curcuminoid, echinacea alkylamides, soy isoflavones, hesperidin, grapeseed extract, milk thistle extract, procyanidins, shizandra berry extract, lemon balm extract, ginger root extract, rhodiola extract, berberine extract, boswellia extract, CoQ10, ubiquinol, vitamin D3, sterols, stanols or any combination thereof.

48. The method of claim 47, wherein the nutraceutical comprises quercetin.

49. The method of claim 38, wherein the lipid-soluble matrix further comprises saponins, cocoa, or any combination thereof.
